(19) United States
(12) Patent Application Publication
(10) Pub. No.: US 2012/0141398 A1
(43) Pub. Date: Jun. 7, 2012

(54) HAIR DYE CONTAINING ONE OR MORE EDIBLE OR COSMETIC COLORS

(76) Inventor: KUO SHENG CHUANG, Taichung City (TW)

(21) Appl. No.: 13/218,443

(22) Filed: Aug. 25, 2011

(30) Foreign Application Priority Data
Dec. 1, 2010 (TW) 099141630

(51) Int. Cl. A61K 8/97; A61Q 5/06; A61Q 5/04; A61K 8/44; A61K 8/46; A61Q 1/04; A61K 8/34; A61K 8/36; A61K 8/42; A61K 8/368; A61K 8/49; A61Q 1/02; A61Q 5/10; A61K 8/22

(52) U.S. Cl. 424/62; 8/405; 424/70.2; 424/70.6; 424/63; 424/64

(57) ABSTRACT

This invention discloses a hair dye and a hair dyeing method using a dye mainly made of natural colors (used in food and cosmetics), synthetic colors (used in food and cosmetics), and cosmetic colors. The hair dye can be applied for dyeing hair and skin at the same time according to the keratin-like structure of the hair and skin. The hair dye can be manufactured in a form of a hair dye cream, a hair dye lotion, a frost hair dye, a hair dye gel, a hair dye ointment, a hair dye shampoo, a foam type hair dye, or a spray type hair dye. After the hair dye is combined with a perm agent, a dyeing and perming product is produced for dyeing and perming hair all at one time. The natural colors (used in food and cosmetics), synthetic colors (used in food and cosmetics) and cosmetic colors of the hair dye penetrate into the hair by different hair dyeing methods to achieve the hair dyeing effect, and permanent hair dye, semi-permanent hair dye and temporary hair dye are manufactured according to the depth that the color enters into the hair structure.

### BACKGROUND OF THE INVENTION

1. Field of the Invention

The present invention relates to a hair dye mainly manufactured by natural colors (used in food and cosmetics), synthetic colors (used in food and cosmetics), and cosmetic colors. The hair dye can be used for dyeing hair and skin according to the keratin-like structure of a user's skin and the hair, and the hair dye can be manufactured in form of a hair dye cream, a hair dye lotion, a frost hair dye, a hair dye gel, a hair dye ointment, a hair dye shampoo, a foam type hair dye or a spray type hair dye. In addition, the hair dye can be combined with a perm agent to produce a two-in-one product for dyeing hair and performing a perm at the same time. The natural colors (used in food and cosmetics), synthetic colors (used in food and cosmetics) or cosmetic colors in the hair dye are permeated into a user's hair by different hair dyeing methods to achieve the hair dyeing purpose, and the hair dye is mainly divided into a permanent hair dye, a semi-permanent hair dye and a temporary hair dye according to how deep the colors can enter into the hair structure.

2. Description of the Related Art

As mentioned above, the hair dye is mainly divided into temporary hair dye, semi-permanent hair dye, and permanent hair dye. The temporary hair dye generally refers to a hair dye that can be removed by shampooing the hair once. Since the particles of the temporary hair dye are relatively larger and cannot enter into a user's hair cortex layer through cuticles, the particles are simply deposited onto the hair surface to form a color covering layer. The semi-permanent hair dye generally refers to a hair dye that can last after 6 to 12 times of shampooing the hair, and the semi-permanent hair dye can be divided into acidic, basic and diversified dyes, wherein the acidic dye is mainly an azo acid dye, and the effect of ionic bonds is used for depositing the dye and showing the color, and the acidic dye can be used in conjunction with a solvent such as benzyl alcohol and N-methylpyrrolidone, and an citric acid can be added to adjust the pH value to improve the hair dyeing effect. The alkaline hair dye is primarily a basic dye or a nitro-amino dye and can be used in conjunction with an alkali expanded cuticle such as triethanolamine, and the semi-permanent hair dye is coated onto the hair, and then rinsed by water after the dye has remained on the hair for 20-30 minutes in order to dye the hair. The permanent hair dye is mainly divided into three types: a permanent metallic hair dye, a permanent botanic hair dye, and a permanent oxidizing hair dye.

(1) Permanent Metallic Hair Dye: a metal salt is used as a raw material for dyeing hair, and the metal salt is deposited onto a surface of hair shafts, such that the hair has a darker luster with a metal appearance, and the hair becomes fragile, and the efficiency of the permanent wave becomes low.

(2) Permanent Botanic Hair Dye: The present permanent botanic hair dye is mainly divided into two types: (I) Color Adsorption: The complexation of the botanic dye with a cation surfactant forms small particles covered onto a hair surface, and other methods include: mixing several kinds of botanic dyes to show different colors. Since dye molecules react with cuticle by the adsorption method only, such dyes are categorized as temporary or semi-permanent hair dyes. (II) Metal Ion Complex: Active ingredients (such as hematoxylin or tannic acid) in a plant and metal ions form a colored complex which penetrates into a hair cortex layer. Since the dye molecules can enter into the hair cortex, such dye is categorized as a permanent hair dye, and the matter of this sort is generally a polyphenol-rich and tannic acid-rich pant, and the representative polyphenol is hematoxylin and the representative tannic acid comes from gall nuts, cloves, and pomegranate peels.

(3) Permanent Chemical Oxidizing Hair Dye: This hair dye is the mainstream product available in the market, wherein the first dose of the chemical oxidizing hair dye has a main composition of an oxidation dye, a coupling agent and an alkali, and the second dose has a main composition of concentrate hydrogen peroxide. In the hair dyeing principle of the chemical oxidizing hair dye, the alkali in the first dose is used for opening the hair cuticles and then the oxidation dye and the coupling agent enter into the hair cortex layer successfully, and then the second dose containing hydrogen peroxide oxidizes the oxidation dye and the coupling agent entered into the hair cortex layer to promote a direct deposition of a polymer dye formed after the oxidation takes place onto the hair cortex layer to achieve a permanent hair dyeing effect. However, latest research reports have indicated that most oxidation dyes and coupling agents are highly toxic and carcinogenic, and slight to severe allergic reactions may occur at the skin easily after a long time of use or an excessive use. Furthermore, the alkali used in the first dose has bad smell and its strong alkalinity (pH>9.5) will damage hair texture seriously, such that after a long time of use or an excessive use, the hair texture will be damaged and the skin will be irritated. Therefore, people play more and more attention to the toxic side effect and the safety issue of the chemical oxidizing hair dye, and developed countries have paid high concerns and conducted researches to the carcinogenicity caused by the chemical oxidizing hair dyes of this sort. At present, many publications (as listed in Table 1) have shown that many oxidation dyes and coupling agents in the chemical oxidizing hair dye are genotoxic, wherein m-phenylenediamine (MPD) is categorized as a suspected carcinogen of animals by World Health Organization and categorized as an induced anaphylactic substance and banned recently by European Union, and Taiwan follows U.S. and Japan and has not banned MPD yet, but limits the content below 5% of the product.

TABLE 1
Mutagencity of Main Composition of Chemical Oxidizing Hair Dye

| | Ames | Other | |
|---|---|---|---|
| Composition | Test | Method | Reference |
| p-nitroaniline | + | + | [3] |
| p-phenylenediamine | + | + | [3] |
| | + | | [2] |
| | | + | [7] |
| o-phenylenediamine | + | + | [3] |
| | + | | [1] |
| 2-nitro-p-phenylenediamine | + | + | [3] |
| | + | | [4, 5] |
| 3-nitro-o-phenylenediamine | + | + | [3] |
| | + | | [5] |
| 4-nitro-o-phenylenediamine | + | + | [3] |
| | + | | [4, 5] |
| 2,4 diaminoanisole | + | | [1, 2, 9] |
| 2,5 diaminoanisole | + | | [1, 10] |
| 2,4 diaminodiphenyl ether | + | | [1] |
| | + | | [2] |
| | + | + | [9] |
| | | + | [10] |
| m-phenylenediamine | + | + | [1, 9, 10] |
| | + | | [2] |
| 2,5 diaminodiphenyl ether | + | | [1, 2] |
| 4-nitro-o-phenylenediamine | + | | [1, 3] |
| | | + | [9] |
| 2-amino-4-nitrophenolate | + | | [1, 10] |
| | | + | [9] |
| 2-amino-5-nitrophenol | | | [1, 10] |
| | | + | [9] |
| p-hydroxyl-m-phenylenediamine | + | | [13] |
| p-methoxy-m-phenylenediamine | + | | [13] |
| 4-amino-3-nitro-6-anisidine | + | | [14] |
| 2,4-diamino-ethoxy benzene | + | | [10, 11] |
| 2,4-diamino-n-propoxyl benzene | + | | [10, 11] |
| 2,4-diamino-ethylbenzene | + | + | [10] |
| | + | | [11] |
| 2,4-diamino-isopropoxyl benzene | + | | [10, 11] |
| | | + | [9] |
| 3-nitro-4-N-β-hydroxyethyl anisidine | + | | [10] |
| 4-amino-3-nitro-phenoxyethanol | + | | [10] |
| 4-nitro-2-amino-6-methylaniline | + | | [12] |
| 4-nitro-2-amino-6β-hydroxyethyl aniline | + | | [12] |
| 4-nitro-2-amino-6β-hydroxypropyl aniline | + | | [12] |
| 4-nitro-2-amino-6β-isopropylaniline | + | | [12] |
| 2-nitro-6-methyl | + | | [12] |
| 2-nitro-6β-hydroxyethyl-p-phenylenediamine | + | | [12] |
| 4-amino-3-nitro-5-methylaniline | + | | [14] |
| 4-amino-3-nitro-5-β-hydroxy-propylaniline | + | | [14] |
| 4-amino-3-nitro-5-isopropylaniline | + | | [14] |
| 4-amino-3-nitro-5,6-dimethylaniline | + | | [14] |
| 4-amino-2,5-dimethylaniline | + | | [14] |

Reference: J Environ Occup Med. 2008, 25. 1, p 100.[Research Development on Mutagenocity, Carcinogenicity and Toxicokinetics of Hair Dyes]

P-phenylenediamine (PPD) is a main allergen in the hair dye, and a necessary chemical composition used in dark brown hair dye (as shown in Tables 2 and 3), and the color content is less, and its median lethal dose is equal to 80 mg/kg indicating a high toxicity of this material, and most hair dyes available in the market, particularly the chemical oxidizing hair dye, contain PPD. Medical professionals indicate that PPD is an allergen and also a carciogen, and thus Germany, France, and Sweden have banned the use of PPD in hair dyes, and the concentration of PPD of the hair dyes sold in the market ranges from 0.01% to 2%, which complies with the limitation of 4% set forth by the Taiwan's regulation, but PPD will cause allergy regardless of the dosage of PPD. Furthermore, the coupling agent in the chemical oxidizing hair dye is toxic, and resorcinol as shown in Table 2 has a median lethal dose equal to 200 mg/kg, and the aforementioned data show a high concern about the toxic side effect and safety of the chemical oxidizing hair dyes.

TABLE 2
List of Toxic Compositions of Black Chemical Oxidizing Hair Dye

| No. | Composition | Median Lethal Dose/Kg) | Carcinogenicity/Allergy | Dosage |
|---|---|---|---|---|
| 1 | p-Phenylenediamine | 80 g (large rat) | Positive Reaction/Causing | 2% |
| | | | Allergic Reaction | |
| 2 | p-Aminophenol | 375 g (small rat) | Causing Allergic Reaction | 0.2% |
| 3 | 3-Aminophenol | 400 g (small rat) | Causing Allergic Reaction | 0.3% |
| 4 | Resorcinol | 200 g (small rat) | Causing allergic reaction | 1.2% |
| 5 | 2-Aminophenol | 800 g (small rat) | Causing allergic reaction | 0.1% |
| 6 | 2-Amino-4- | No toxic data | Causing allergic reaction | 0.8% |
| | hydroxyethylaminoanisole | | | |
| | sulfate | | | |
| 7 | Ammonia solution | | Causing allergic reaction | 3% |

TABLE 3
List of Toxic Compositions of Brown Chemical Oxidizing Hair Dye

| No. | Composition | Median Lethal Dose/Kg) | Carcinogenicity/Allergy | Dosage |
|---|---|---|---|---|
| 1 | p-Phenylenediamine | 80 g (large rat) | Positive Reaction/Causing | 0.5% |
| | | | Allergic Reaction | |
| 2 | N,N-Bis(2-hydroxyethyl)- | 131 g (large rat) | Causing Allergic Reaction | 0.05% |
| | p-phenylenediamine | | | |
| | sulfate | | | |
| 3 | 3-Aminophenol | 400 g (small rat) | Causing Allergic Reaction | 0.01% |
| 4 | 4-Chlororesorcinol | 369 g (large rat) | Causing Allergic Reaction | 0.48% |
| 5 | 2-amino-4- | No toxic data | Causing Allergic Reaction | 0.05% |
| | hydroxyethylaminoanisole | | | |
| | sulfate | | | |
| 6 | Ammonia solution | | | 7% |

Although the toxicity of semi-permanent hair dyes and temporary hair dyes has no toxic side effect of the permanent chemical oxidizing hair dye, yet the discoloration level is quick and the dye is not wash-resisting, and its use still does not have the advantages of the permanent hair dye, and the botanic hair dye regardless of the adsorption or metal complex type has at least two drawbacks: a complicated using procedure and too little colors. Although the botanic hair dye is relatively safe, it still cannot replace the popularity of the chemical hair dyes.

The primary objective of the present invention is to overcome the aforementioned drawbacks of the conventional hair dyeing systems by providing a safe, wash-resisting permanent dyeing system for people who need to dye their hair, and the present invention provides a novel fast hair dyeing method for dyeing hair and improves the allergy, safety and applicability of the conventional hair dyes.

### SUMMARY OF THE INVENTION

The present invention relates to a hair dye primarily made of a natural colors (used in food and cosmetics), a synthetic colors (used in food and cosmetics), or a cosmetic colors and a hair dyeing method of manufacturing the hair dye. The hair dye can be applied for dyeing hair and skin according to the keratin-like structures of the skin and the hair. The hair dye can be manufactured into a hair dye cream, a hair dye lotion, a frost hair dye, a hair dye gel, a hair dye ointment, a hair dye shampoo, a foam type hair dye, or a spray type hair dye. After the hair dye is combined with a perm agent, a two-in-one product is manufactured for dyeing and having a perm simultaneously. The hair dye achieve the hair dyeing purpose by penetrating and entering the natural colors (used in food and cosmetics), synthetic colors (used in food and cosmetics), or cosmetic colors by different hair dyeing methods, and permanent hair dyes, semi-permanent hair dyes and temporary hair dyes are manufactured according to the depth that colors penetrate into the hair structure, and the hair dyeing methods include a metal mordant dyeing method, a disulfide bond reduction method, an oxidation method, an ionic bond method and an adsorption method.

The colors of the present invention refer to natural colors (used in food and cosmetics), synthetic colors (used in food and cosmetics), and cosmetic colors as shown in Tables 4, 5 and 6. The natural colors and food colors are edible colors without any safety concern, and the cosmetic colors are not edible, yet they are used as color additives of cosmetic products, so that its safety of applying to skin and hair is higher than the organic dye among the hair dyes. The colors of the present invention include the main colors such as red, yellow, blue, pink, green, orange, tangerine, purple, black and white (as listed in Tables 4, 5, and 6), and one color with its hair dyeing method can be used for dyeing hair, or different colors mixed with different proportions in conjunction with its hair dyeing method can be used for dyeing hair, or a color formed by mixing the three primitive colors: red, yellow and blue with different proportions and used in conjunction with its hair dyeing method can be used for dyeing hair. The method of producing the color of a dye is similar to the principle of mixing colors. Before hair is dyed, dyes of different colors are mixed. When a hair dye product of a color is produced, different colors in a predetermined proportion are mixed to obtain the hair dye product. The hair dye colors of the present invention cover all colors and can be prepared before dyeing hair or when producing the hair dye and the present invention is not limited to any particular arrangement.

TABLE 4
List of Natural Food Colors

| | | Main |
|---|---|---|
| Colors | Plant | Composition |
| Plum Colors | Plum | Anthocyanins |
| Purple Corn Colors | Maiz morado | Anthocyanins |
| Red Cabbage Colors | Brassica oleracea L. var. | Anthocyanins |
| | Capitata DC. ev. Red Acre | |
| Blueberry Color | Vaccinium corymbosum L. | Anthocyanins |
| Cherry Colors | Prunus pauciflra BUNCH | Anthocyanins |
| Grape Juice Colors | Vitis vinifera L. | Anthocyanins |
| Grape Skin Colors | Red grapes | Anthocyanins |
| Hibiscus Colors | Hibiscus sabdariffa L. | Anthocyanins |
| Mulberry Colors | Morus nigra L., M. alba L. | Anthocyanins |
| Perilla Colors | Perilla | Anthocyanins |
| Strawberry Colors | Fragaria ananassa | Anthocyanins |
| | DUCHESNE | |
| Aronia Berries Colors | Prunus viginiana | Anthocyanins |
| Blackcurrant Fruit Colors | Ribes nigrum | Anthocyanins |
| Black Carrot Colors | Daucus carota | Anthocyanins |
| Elderberries Colors | Elderberries | Anthocyanins |
| Gardenia Blue | Gardenia | Genipin |
| Gardenia Yellow | Gardinia augusta MERR. | Crocin |
| | vargracliflora HORT | |
| Annatto, water or oil | Bixa orellana L. | Norbixin; Bixin |
| soluble | | |
| Amaranthus Colors | Red amaranth | Amaranthin |
| Cocoa Colors | Theobro macacao | Flavonoids |
| Licorice Colors | Glycyrrhiza glabra L., | Flavonoids |
| | Glycyrrhiza uralensis FISCH. | |
| Buckwheat Extract | Fagopyrum esculentum | Flavonoids |
| | MOENCH | |
| Saffron | Crocus sativus L. | Flavonoids |
| Safflower Yellow | Carthamus tinctorius | Flavonoids |
| Onion Colors | Allium cepa L. | Flavonoids |
| Peanut Colors | Arachis hypogaea L. | Flavonoids |
| Sorghum Colors | Sorghum | Flavonoids |
| Caramel | Sugar | Sugar |
| Sugar Cane Colors | Sugar | Sugar |
| Malted Barley Colors | Malted barley | Sugar |
| Turmeric | Curcuma longa | Curcumin |
| Carmine | Coccus cacti L. | Carminic Acid |
| Laver Colors | Porphyra tenera KJELLM. | Phycoerythrin |
| Spirulina Colors | Spirulina | Phycocyanin |
| Monascus Colors | Monascus purpureus, | |
| | Monascusanka | |
| Beet Red | Beta vulgaris | Betanin |
| Tamarind Color | Tamarindus indica L. | Polyphenol |
| Persimmon Colors | Persimmon | Polyphenol |
| Chlorella Colors | Chlorella | Chlorophyll |
| Chlorophyll Colors | Green plant | Chlorophyll |
| Stinging Nettle Colors | Urtica dioica | Chlorophyll |
| Spinach Colors | Soinacia oleracea | Chlorophyll |
| Carrot Colors | Daucus carota subsp. Sativus | β-Carotene |
| Sweet Potato Colors | Ipomoea batatas POIR. | β-Carotene |
| Corn Colors | Zea mays L. | Carotenoids |
| Crawfish Colors | Crab | Carotenoids |
| Orange Colors | Orange | Carotenoids |
| Paprika Colors | Caprium annuum | Carotenoids |
| Shrimp Colors | Shrimp | Carotenoids |
| Tomato Colors | Tomato | Lycopene |
| Aztec Marigold Colors | Aztec marigold | Xanthophylls |
| Xanthophylls | Alfalfa | Xanthophylls |
| SandalWood Colors | Pterocarpus santalinus) | Santalin |
| Sepiomelanin Colors | Speia officinalis | Sepiomelanin |

TABLE 5
List of Food Synthetic Colors

| | | | | | Names of |
|---|---|---|---|---|---|
| | | | | | Food Colors |
| | | | | Names of Food Colors | Used in |
| No. | CI No. | CAS No. | Colors | Used in U.S.A. | Taiwan |
| 1 | 12156 | 6358-53-8 | Citrus Red No. 2 | Citrus Red No. 2 | |
| 2 | 15985 | 2783-94-0 | FD&C Yellow No. 6 | FD&C Yellow No. 6 | Taiwan Food |
| | | | | | Yellow No. 5 |
| 3 | 15985:1 | 15790-07-5 | FD&C Yellow No. 6 | FD&C Yellow No. 6 | Taiwan Food |
| | | | Lakes | Lakes | Yellow No. 5 |
| 4 | 16035 | 25956-17-6 | FD&C Red No. 40 | FD&C Red No. 40 | Taiwan Food |
| | | | | | Red No. 40 |
| 5 | 16035:1 | 68583-95-9 | FD&C Red No. 40 | FD&C Red No. 40 | Taiwan Food |
| | | | Lakes | Lakes | Red No. 40 |
| 6 | 16255 | 2611-82-7 | Acid Red 18 | | Taiwan Food |
| | | | | | Red No. 6 |
| 7 | 19140 | 1934-21-0 | FD&C Yellow No. 5 | FD&C Yellow No. 5 | Taiwan Food |
| | | | | | Yellow No. 4 |
| 8 | 19140:1 | 12225-21-7 | FD&C Yellow No. 5 | FD&C Yellow No. 5 | Taiwan Food |
| | | | Lakes | Lakes | Yellow No. 4 |
| 9 | 19235 | 15139-76-1 | Orange B | Orange B | |
| 10 | 42053 | 2353-45-9 | FD&C Green No. 3 | FD&C Green No. 3 | Taiwan Food |
| | | | | | Green No. 3 |
| 11 | 42090 | 3844-45-9 | FD&C Blue No. 1 | FD&C Blue No. 1 | Taiwan Food |
| | | | | | Blue No. 1 |
| 12 | 42090:2 | 68921-42-6 | FD&C Blue No. 1 | FD&C Blue No. 1 | Taiwan Food |
| | | | Lakes | Lakes | Blue No. 1 |
| 13 | 45430 | 16423-68-0 | FD&C Red No. 3 | FD&C Red No. 3 | Taiwan Food |
| | | | | | Red No. 7 |
| 14 | 45430:1 | 12227-78-0 | FD&C Red No. 3 | FD&C Red No. 3 | Taiwan Food |
| | | | Lakes | Lakes | Red No. 7 |
| 15 | 73015 | 860-22-0 | FD&C Blue No. 2 | FD&C Blue No. 2 | Taiwan Food |
| | | | | | Blue No. 2 |
| 16 | 73015:1 | 16521-38-3 | FD&C Blue No. 2 | FD&C Blue No. 2 | Taiwan Food |
| | | | Aluminum Lake | Aluminum Lake | Blue No. 2 |

TABLE 6
List of Cosmetic Colors

| No. | CI No. | CAS No. | Colors |
|---|---|---|---|
| 1 | 10020 | 19381-50-1 | Ext. D&C Green No. 1 |
| 2 | 10316 | 846-70-8 | Ext. D&C Yellow No. 7. |
| 3 | 11380 | 85-84-7 | Ext. D&C Yellow No. 9 |
| 4 | 11390 | 131-79-3 | Ext. D&C Yellow No. 10 |
| 5 | 11680 | 2512-29-0 | Ext. D&C Yellow No. 5 |
| 6 | 11710 | 6486-23-3 | Pigment Yellow 3 |
| 7 | 11725 | 6371-96-6 | Ext D and C Orange No. 1 |
| 8 | 11743 | 12236-75-8 | Pigment Yellow 105 |
| 9 | 12085 | 2814-77-9 | D&C Red No. 36. |
| 10 | 12010 | 6535-42-8 | Solvent Red 3 |
| 11 | 12100 | 2646-17-5 | Ext. D&C Orange No. 4 |
| 12 | 12120 | 2425-85-6 | D&C Red No. 35 |
| 13 | 12140 | 3118-97-6 | Ext. D&C Red No. 14 |
| 14 | 12150 | 1229-55-6 | Solvent Red 1 |
| 15 | 12156 | 6358-53-8 | Citrus Red No. 2 |
| 16 | 12315 | 6448-95-9 | Pigment Red 22 |
| 17 | 12490 | 6410-41-9 | Pigment Red 5 |
| 18 | 13015 | 2706-28-7 | Acid Yellow 9 |
| 19 | 13058 | 6371-55-7 | D&C Red No. 39. |
| 20 | 13065 | 587-98-4 | Ext. D& C Yellow No. 1 |
| 21 | 14600 | 523-44-4 | Ext. D&C Orange No. 3 |
| 22 | 14700 | 4548-53-2 | FD&C Red No. 4 |
| 23 | 14720 | 3567-69-9 | Ext D & C Red No. 10 |
| 24 | 15510 | 633-96-5 | D&C Orange No. 4. |
| 25 | 15585:1 | 5160-02-1 | D&C Red No. 9 |
| 26 | 15620 | 1658-56-6 | Ext. D&C Red No. 8 |
| 27 | 15630 | 1248-18-6 | D&C Red No. 10 |
| 28 | 15630:1 | 1103-38-4 | D&C Red No. 12 |
| 29 | 15630:2 | 1103-39-5 | D&C Red No. 11 |
| 30 | 15630:3 | 6371-67-1 | D&C Red No. 13 |
| 31 | 15800 | 6371-76-2 | D&C Red No. 31. |
| 32 | 15850 | 5858-81-1 | D&C Red No. 6. |
| 33 | 15850:1 | 5281-04-9 | D&C Red No. 7 |
| 34 | 15850:2 | 17852-98-1 | D&C Red No. 6, barium lake |
| 35 | 15865 | 3564-21-4 | Pigment Red 48 |
| 36 | 15880 | 21416-46-6 | D&C Red No. 34. |
| 37 | 15880:1 | 6417-83-0 | D&C Red No. 34 |
| 38 | 15985 | 2783-94-0 | FD&C Yellow No. 6 |
| 39 | 15985:1 | 15790-07-5 | FD&C Yellow No. 6 Lakes |
| 40 | 16035 | 25956-17-6 | FD&C Red No. 40 |
| 41 | 16035:1 | 68583-95-9 | FD&C Red No. 40 Lakes |
| 42 | 16150 | 3761-53-3 | D&C Red No. 5 |
| 43 | 16155 | 3564-09-8 | FD&C Red No. 1 |
| 44 | 16185 | 915-67-3 | FD&C Red No. 2 |
| 45 | 16185:1 | 12227-62-2 | FD&C Red No. 2 Lakes |
| 46 | 16255 | 2611-82-7 | Acid Red 18 |
| 47 | 16255:1 | 12227-64-4 | Pigment Red 274 |
| 48 | 17200 | 3567-66-6 | D&C Red No. 33. |
| 49 | 18050 | 3734-67-6 | Ext D and C Red No. 11 |
| 50 | 18820 | 6359-82-6 | Ext. D& C Yellow No. 3 |
| 51 | 18950 | 6372-96-9 | Ext. D&C Yellow No. 4 |
| 52 | 19140 | 1934-21-0 | FD&C Yellow No. 5 |
| 53 | 19140:1 | 12225-21-7 | FD&C Yellow No. 5 Lakes |
| 54 | 19235 | 15139-76-1 | Orange B |
| 55 | 20170 | 1320-07-6 | D&C Brown No. 1. |
| 56 | 20470 | 1064-48-8 | D&C Black No. 1. |
| 57 | 21090 | 6358-85-6 | Pigment Yellow 12 |
| 58 | 21110 | 3520-72-7 | Pigment Orange 13 |
| 59 | 26100 | 85-86-9 | D&C Red No. 17. |
| 60 | 26105 | 85-83-6 | Solvent Red 24 |
| 61 | 42052 | 3374-30-9 | D&C Blue No. 7 |
| 62 | 42053 | 2353-45-9 | FD&C Green No. 3 |
| 63 | 42085 | 4680-78-8 | FD&C Green No. 1 |
| 64 | 42090 | 3844-45-9 | FD&C Blue No. 1 |
| 65 | 42090 | 2650-18-2 | D&C Blue No. 4 |
| 66 | 42090:1 | 6548-12-5 | Pigment Blue 24 |
| 67 | 42090:2 | 68921-42-6 | FD&C Blue No. 1 Lakes |
| 68 | 42095 | 5141-20-8 | D&C Green No. 4 |
| 69 | 42640 | 1694-09-3 | FD&C Violet No. 1 |
| 70 | 45100 | 3520-42-1 | Acid Red 52 |
| 71 | 45160:1 | 12224-98-5 | Pigment Red 81 |
| 72 | 45170:3 | 12227-77-9 | Pigment Red 173 |
| 73 | 45190 | 6252-76-2 | Ext. D&C Red. No3 |
| 74 | 45350 | 518-47-8 | D&C Yellow No. 8. |
| 75 | 45350:1 | 2321-07-5 | D&C Yellow No. 7. |
| 76 | 45370:1 | 596-03-2 | D&C Orange No. 5. |
| 77 | 45370:2 | 93776-20-6 | Pigment Orange 39 |
| 78 | 45380 | 17372-87-1 | D&C Red No. 22. |
| 79 | 45380:2 | 15086-94-9 | D&C Red No. 21. |
| 80 | 45380:3 | 15876-39-8 | Pigment Red 90:1 |
| 81 | 45410 | 18472-87-2 | D&C Red No. 28. |
| 82 | 45410:1 | 2134-15-8 | D&C Red No. 27. |
| 83 | 45410:2 | 15876-58-1 | Pigment Red 174 |
| 84 | 45425 | 33239-19-9 | D&C Orange No. 11. |
| 85 | 45425:1 | 38577-97-8 | D&C Orange No. 10. |
| 86 | 45430 | 16423-68-0 | FD&C Red No. 3 |
| 87 | 45430:1 | 12227-78-0 | FD&C Red No. 3 Lakes |
| 88 | 45440 | 11121-48-5 | Acid Red 94 |
| 89 | 47000 | 8003-22-3 | D&C Yellow No. 11. |
| 90 | 47005 | 8004-92-0 | D&C Yellow No. 10. |
| 91 | 47005:1 | 68814-04-0 | D&C Yellow No. 10. Lakes |
| 92 | 51319 | 6358-30-1 | Pigment Violet 23 |
| 93 | 58000:1 | 104074-25-1 | Pigment Red 83 |
| 94 | 59040 | 6358-69-6 | D&C Green No. 8. |
| 95 | 60725 | 81-48-1 | D&C Violet No. 2. |
| 96 | 60730 | 4430-18-6 | Ext. D&C Violet No. 2. |
| 97 | 61520 | 6408-50-0 | Solvent Blue 63 |
| 98 | 61565 | 128-80-3 | D&C Green No. 6. |
| 99 | 61570 | 4403-90-1 | D&C Green No. 5. |
| 100 | 69825 | 130-20-1 | D&C Blue No. 9. |
| 101 | 73000 | 482-89-3 | D&C Blue No. 6. |
| 102 | 73015 | 860-22-0 | FD&C Blue No. 2 |
| 103 | 73015:1 | 16521-38-3 | FD&C Blue No. 2 Aluminum Lake |
| 104 | 73360 | 2379-74-0 | D&C Red No. 30. |
| 105 | 74160 | 147-14-8 | Pigment Blue 15 |
| 106 | 74260 | 1328-53-6 | Pigment Green 7 |
| 107 | 74350 | 1328-54-7 | Solvent Blue 25 |
| 108 | 75170 | 73-40-5 | Natural White 1 |
| 109 | 77019 | 12001-26-2 | Mica-based pearlescent colors |
| 110 | 77266 | 1333-86-4 | D&C Black No. 2 |

The hair dyeing methods of the present invention include a metal mordant dyeing method, a disulfide bond reduction method, an oxidation method, an ionic bond method and an adsorption method, and the properties of each method are listed in Table 7.

TABLE 7
Properties of Hair Dyes of the Present Invention

| | Hair Dye Type | | |
|---|---|---|---|
| Definition | Temporary Hair Dye | Semi-permanent Hair Dye | Permanent Hair Dye |
| Dyeing Position | Hair and Skin | | |
| Depth of Dye | Cuticle | Cuticle and Part of Hair | Hair Cortex |
| Penetrating into Hair | | Cortex | |
| No. of Times of | 1 time | 6-12 times | 30 times |
| Shampoos that the | | | |
| Dye Lasts | | | |
| No. of Days that the | 1 day | 15 days | 30 days |
| Dye Lasts | | | |
| Types of Colors Used | Three types of colors including natural colors (used in food and | | |
| | cosmetics), synthetic colors (used in food and cosmetics) and cosmetic | | |
| | colors can be used. | | |
| Dosage of Hair Dye | Hair dye cream, hair dye lotion, frost hair dye, hair dye gel, hair dye | | |
| Used | ointment, hair dye shampoo, foam type hair dye, spray type hair dye, and | | |
| | dyeing and perming type | | |
| Color of Hair Dye | Any color | | |
| Hair dyeing method | adsorption | Ionic bond method | Oxidation |
| properties | | | metal mordant |
| | | | dyeing |
| | | | Disulfide bond |
| | | | reduction |

The metal mordant dyeing method uses a high-priced metal ions as an intermediate coupling agent of the protein of the hair cortex layer and the colors of the hair dye for producing a sandwich interaction, so that the colors can be fixed into the hair cortex layer to achieve the hair dyeing purpose, and this hair dyeing method can be applicable for hair dye products manufactured by natural colors (used in food and cosmetics), synthetic colors (used in food and cosmetics) and cosmetic colors. The metal ions include ions of magnesium, aluminum, iron and zinc salts, and the action force includes action forces of covalent bond and ionic bond.

The disulfide bond reduction is a method for achieving a hair dyeing purpose by using a reducer to break the disulfide bond of hair proteins to open the hair cuticle, such that color molecules can penetrate into the hair cortex layer, while hair penetration enhancer and hair expander can be added to improve the dyeing performance, and the colors applicable for this hair dyeing method include hair dye products manufactured by natural colors (used in food and cosmetics), synthetic colors (used in food and cosmetics) and cosmetic colors.

In the oxidation, hydrogen peroxide is used for discoloring the black color of hair by oxygen produced in a weak alkali (pH=7.5-8.5), while the weak alkali can open the cuticle, such that color molecules can enter into the hair cortex layer. With the aforementioned double effects, a safe method without harming the hair texture can deposit colors onto the cortex layer to achieve the hair dyeing purpose. This method is substantially similar to the method of operating the permanent chemical oxidizing hair dye, but the operating method is not exactly the same, wherein the hydrogen peroxide in the chemical hair dye has the functions of discoloring the black color of hair, and forming dye molecules from the intermediate of the chemical oxidizing dye, and the hydrogen peroxide used in this method can produce oxygen only to discolor the black color of hair, so that this method simply requires low-concentration hydrogen peroxide without the need of a concentrate hydrogen peroxide, and the damage to the hair texture can be reduced. In addition, the present invention uses a weak alkali (pH=7.5-8.5) to drive the hydrogen peroxide to produce oxygen and open the hair cuticle in order to facilitate the color molecules to enter into the hair cortex layer without damaging the hair texture or lowering the dyeing performance, and the chemical hair dye must use a strong base (pH>9.5) to achieve the effects of opening the cuticle and promoting the production of oxygen from hydrogen peroxide. Under the environment of a strong base, many amino acids of hair proteins will have a chemical reaction with the strong base, so that any strong base (pH>9.5) will have severe damages to our hair. In the present invention, this hair dyeing method is applicable to hair dye products manufactured by colors including natural colors (used in food and cosmetics), synthetic colors (used in food and cosmetics) and cosmetic colors. Wherein, the reagent used in the oxidation is a compound capable of producing oxygen and including the reagent such as inorganic peroxide and organic peroxide and capable of producing oxygen. Wherein, the alkali is an organic alkali or an inorganic alkali.

In the ionic bond method, anions or cations of the colors and the hair protein produce an action force of ionic bonds that operates in conjunction with a hair expander and a penetration enhancer to deposit the colors on the hair cortex or in a middle area between the cuticle and the hair cortex to achieve the hair dyeing purpose. In addition, an acid can be added to the colors containing anions to adjust its pH value to improve the hair dyeing effect; and an alkali can be added to the colors containing cations to adjust its pH value to improve the hair dyeing effect. The hair dyeing method of the present invention is applicable to hair dye products manufactured by colors such as the natural colors (used in food and cosmetics), synthetic colors (used in food and cosmetics) and cosmetic colors containing anions or cations. Wherein, the acid is an organic acid or an inorganic acid, and the alkali is an organic alkali or an inorganic alkali.

In the adsorption method, a simple adsorption between color molecules and hair proteins is used to coat the colors onto a hair surface to form a color covering layer to achieve the hair dyeing purpose. The hair dyeing method of the present invention is applicable for hair dye product manufactured by colors including natural colors (used in food and cosmetics), synthetic colors (used in food and cosmetics) and cosmetic colors.

The present invention discloses a hair dye manufactured by natural colors (used in food and cosmetics), synthetic colors (used in food and cosmetics) and cosmetic colors and a hair dyeing method thereof, and successfully uses non-toxic natural colors and food colors as the raw material for dyeing hair to overcome the long-existing problems of the convention hair dyes with genotoxicity, carcinogenicity and allergy, so that the hair dye products enter into a revolutionary stage. In addition to the safety of the dye, the present invention further break through the technology of penetrating the natural colors (used in food and cosmetics), synthetic colors (used in food and cosmetics) and cosmetic colors into the hair to assure that the innovative hair dyeing materials and technologies can replace the long-time habits and advantages, in hope of achieving the objective of providing very safe natural colors (used in food and cosmetics), synthetic colors (used in food and cosmetics) and cosmetic colors for dyeing hair permanently, semi-permanently and temporarily. Therefore, the present invention aims at the technologies of penetrating the colors to different depths of the hair and develops a total of five hair dyeing skills to achieve the aforementioned objective, and these five skills include a metal mordant dyeing method, a disulfide bond reduction method, an oxidation method, an ionic bond method and an adsorption method. Research results indicate that the foregoing objective can be achieved. Now, the next issue is to assure the colors of the hair dye of the present invention can meet the market requirements, wherein the present invention mixes dyes of the three primitive colors: red, yellow and blue as well as black and white colors. Among the mixing methods, single-color dye products are mixed with a predetermined proportion, and then the hair is dyed by a predetermined hair dyeing method, and this method is similar to the method of mixing drawing paints, or the hair dye product is produced by mixing a specific proportion for producing a special-color hair dye, and then the hair is dyed by a predetermined hair dyeing method. In the aforementioned two methods used for producing the colors of the color dyes mix five colors: red, yellow, blue, black and white colors or even other colors to create the colors in accordance with market requirements, and this mixing method is better and more convenient than those of the chemical hair dye. Obviously, the hair dye of the invention has advantages over the chemical hair dye.

Now, the issue whether or not the hair dye can last in a level up to the market standard is taken into consideration. Each type of hair dyes of the present invention has taken strict shampoo tests, and the standards listed in Table 7 (sustainability and wash persistency standards) are used to determine whether the dye is a permanent, semi-permanent or temporary hair dye, and results show that the properties of the hair dye of the present invention are related to the hair dyeing method and the dye formula, wherein the hair dye is categorized as the temporary hair dye if the adsorption method is used for the hair dyeing experiments, and there is only a weak adsorption force between the colors and the hair, and all natural colors (used in food and cosmetics), synthetic colors (used in food and cosmetics) and cosmetic colors of the present invention fall within the category of this type of hair dyes.

If the ionic bond method is used for the hair dyeing experiment, since the hair expander, penetration enhancer and acid and base are added in the formula and steam is used for heating the hair to accelerate the speed and increase the depth of its penetration into the hair, such that a portion of the colors is deposited onto the area of the hair cortex or cuticle, and the dyes using this formula is categorized as the semi-permanent hair dye, since there is a stronger force of the ionic bonds between the colors and the hair, and the natural colors (used in food and cosmetics), synthetic colors (used in food and cosmetics), and cosmetic colors having anions or cations of the present invention fall within the range of this type of hair dyes.

If the disulfide bond reduction method is used for the hair dyeing experiment and the dye formula contains a reducer including broken disulfide bonds of the hair protein, and a weak alkali or steam used for heating the hair physically opens the hair cuticle, then the color molecules can be penetrated and deposited into the hair cortex layer, and thus the dye using this formula and this hair dyeing method is categorized as the permanent hair dye, since the colors are deposited into the hair cortex layer. All natural colors (used in food and cosmetics), synthetic colors (used in food and cosmetics) and cosmetic colors of the present invention fall within the category of this type of hair dyes.

If the oxidation method is used for the hair dyeing experiment, the hair dyeing method mixes hydrogen peroxide, a weak alkali and color dyes, and then uses the mixed ointment for dyeing hair. In the aforementioned environment, oxygen in the ointment will discolor the black color of the hair in the discoloration process, but will not oxidize the colors in the dye. In the environment of a weak alkali, the cuticle will be opened to drive color molecules to enter and deposit into the hair cortex layer, and the dye using this formula and the foregoing hair dyeing method fall within the category of a permanent hair dye, since the colors are deposited into the hair cortex layer, and all natural colors (used in food and cosmetics), synthetic colors (used in food and cosmetics) and cosmetic colors of the present invention fall within the category of this type of hair dye.

If the metal mordant dyeing method is used for the hair dyeing experiment, and the low-toxicity high-priced metal ions are used as a medium and the covalent bonds and ionic bonds of the color molecules are used to achieve the effect of fixing and depositing the color molecules into the hair cortex layer, and the dye using this formula and the foregoing hair dyeing method fall within the category of the permanent hair dye, since the colors are deposited into the hair cortex layer, and all natural colors (used in food and cosmetics), synthetic colors (used in food and cosmetics) and cosmetic colors having anions, cations, and ligands of NH₂ groups, OH groups and SH group of the present invention fall within the category of this type of hair dye.

The final issue remained to be solved is to provide a chemical hair dye with a fast dyeing speed to meet the requirement of dyeing hair in a short time. In the hair dyes of the present invention, the dye using the metal mordant dyeing method takes 40 minutes to dye hair, and the dyes of the present invention using other hair dyeing methods just takes 20-30 minutes to complete the process of dyeing hair. Obviously, the dyeing speed is equivalent to that of the chemical hair dye.

In summation, the hair dye of the present invention has the following advantages:

(1) Highly safe natural colors (used in food and cosmetics), synthetic colors (used in food and cosmetics) and cosmetic colors are used to substitute the genotoxic, carcinogenic and allergic chemical hair dye to achieve the same permanent hair dye standard as the chemical hair dye.

(2) Highly safe natural colors (used in food and cosmetics), synthetic colors (used in food and cosmetics) and cosmetic colors are used to substitute the semi-permanent and temporary hair dyes available in the market to achieve the same hair dye standard.

(3) The colors of the hair dye are prepared by similar color mixing methods, and any color of the hair dye can be prepared, and a predetermined hair dyeing method is used for dyeing hair conveniently and creatively.

(4) All hair dyeing methods and colors used in the present invention primarily take human safety and hair texture into consideration, and a highly safe hair dye not damaging the hair texture is provided.

(5) The hair dyeing process can be completed within 20-30 minutes (except the metal mordant dyeing method requires 40 minutes), and a permanent, semi-permanent or temporary hair dye can be selected for a user's hair dyeing according to the type of color, the dye formula and the hair dyeing method.

(6) The natural colors (used in food and cosmetics), synthetic colors (used in food and cosmetics) and cosmetic colors of the present invention can be manufactured as a product in the form of a hair dye cream, a hair dye lotion, a frost hair dye, a hair dye gel, a hair dye ointment, a hair dye shampoo, a foam type hair dye, a spray type hair dye, or a dyeing and perming dye according to the user requirements.

### BRIEF DESCRIPTION OF THE DRAWINGS

No drawing is provided in this document.

### DETAILED DESCRIPTION OF THE PREFERRED EMBODIMENTS

The present invention discloses a hair dye primarily made of natural colors (used in food and cosmetics), synthetic colors (used in food and cosmetics) and cosmetic colors, and provides a hair dye product applied with different hair dyeing methods to achieve a hair dyeing purpose, and the hair dye can be applied for dyeing hair and skin. The hair dye can be manufactured in form of a hair dye cream, a hair dye lotion, a frost hair dye, a hair dye gel, a hair dye ointment, a hair dye shampoo, a foam type hair dye, and a spray type hair dye. After the hair dye is combined with a perm agent, a dyeing and perming product provided for dyeing and perming hair can be achieved. The hair dye can be manufactured as a permanent hair dye, a semi-permanent hair dye or a temporary hair dye according to the hair dyeing method, the type of colors, and the formula of the hair dye as follows.
1. The types of natural colors (used in food and cosmetics), synthetic colors (used in food and cosmetics) and cosmetic colors are described below:

The natural colors (used in food and cosmetics), synthetic colors (used in food and cosmetics) and cosmetic colors of the present invention include edible natural colors, synthetic colors and cosmetic colors, and useful examples of the edible natural colors are listed in Table 4, and useful examples of the edible synthetic colors are listed in Table 5, and useful examples of the cosmetic colors are listed in Table 6. Since the names of the edible colors and cosmetic colors have a slight difference, therefore the present invention adopts the international Color Index Number (C.I. No.) to represent the commonly used dyes. For colors not indexed, the CAS Number is used for representing the color, or the name of the color is used for representing the color directly.
2. The hair dyeing methods of the natural colors (used in food and cosmetics), synthetic colors (used in food and cosmetics) and cosmetic colors are described in details below:

### (1) Oxidation

The hair dyeing method of the present invention using an oxidation is similar to the method of dyeing hair by a chemical hair dye, and the hair dye product contains a first dose of a color which is primarily a dye ointment, a second dose of the color which is primarily a hydrogen peroxide or peroxide ointment, and a third dose which is primarily an alkaline ointment; and the alkali can be contained in the color ointment, and the hair dyeing procedure comprises the following steps.
1. The dye ointment, hydrogen peroxide or peroxide ointment, and alkaline ointment are mixed in a predetermined proportion and prepared for use.
2. The mixed hair dye is brushed and coated uniformly onto each hair and stays on the hair for 20-30 minutes, and then the dye on the hair is washed by warm water, and then the hair is shampooed and rinsed to complete the hair dyeing process.

In this hair dyeing method of the present invention, the applicable colors include all natural colors (used in food and cosmetics), synthetic colors (used in food and cosmetics), and cosmetic colors, and the type of hair dye is the permanent hair dye, wherein the peroxide refers to a compound that can produce oxygen, and includes inorganic peroxide, organic peroxide and any reagent that can produce oxygen. The inorganic peroxide is an inorganic compound including a peroxy group (—O—O—), an inorganic acid, and their salts, such as hydrogen peroxide, sodium peroxide, potassium peroxide, calcium peroxide, magnesium peroxide, potassium peroxymono sulfate, monopersulfate, dipersulfate, pernitrate, monoperphosphate, diperphosphate, sodium percarbonate, sodium perborate and their salts. The organic peroxide is an organic compound containing a peroxy-chain group (—O—O—) with a general formula of R—O—O—R′, such as peracid, perester, peroxide, ether peroxide, tetrahydrofuran peroxide, ethylene glycol dimethyl ether peroxide, benzoyl peroxide, methyl ethyl ketone peroxide, triacetone triperoxide, peroxyacetyl nitrate, or dioxirane, wherein examples of the peroxy acid include performic acid, peroxyacetic acid, trifluoroperacetic acid, perpropionic acid, perbutanoic acid, perisopentanoic acid, long-chain perfatty acid, peroxybenzoic acid, chloroperoxybenzoic acid, nitroperoxybenzoic acid, and monoperphthalic acid. Other oxygen producing reagents can be ozone or any other similar substance. The alkali includes an organic alkali and an inorganic alkali, and practical examples include alkylamines such as monoethanolamine, diethanolamine, triethanolamine, monopropanolamine, dipropanolamine, tripropanolamine, 2-amino-2-methyl-1,3-propanediol, 2-amino-2-methyl-l-propanol and 2-amino-2-hydromethyl-1,3- propanediol; inorganic salts such as sodium hydroxide, sodium carbonate, sodium bicarbonate, calcium carbonate and calcium bicarbonate; and guanidine salts such as guanidine hydrochloride, guanidine sulfate, guanidine nitrate, guanidine phosphate, guanidine phosphate sulfate , guanidine borate, aminoguanidine sulfate, and the guanidine salts of organic acids, and persons skilled in the art should know that any equivalent peroxides and alkalis can be used instead.

### (2) Disulfide Bond Reduction

In the first hair dyeing method of the present invention adopting the disulfide bond reduction, the colors contained in the product include a first-dose dye ointment, and a second-dose ointment which is primarily a disulfide bond reducer, and an alkali may or may not be added into this dose depending on the properties of the disulfide bond reducer, and the hair dyeing procedure is as follows.
1. The ointment (disulfide bond reducer) is coated uniformly onto each hair by a hair dyeing brush and stays on the hair for 10 minutes, and then a hair dryer or a steamer can be used for heating the hair during the stay period, and this ointment on the hair can be washed away by warm water or the following step is carried out without washing the hair.
2. A color dye ointment is coated uniformly onto each hair by a hair dyeing brush, and stays on the hair for 20 minutes, and then a hair dryer or a steamer can be used for heating the hair during the stay period, and his ointment on the hair can be washed away by warm water, and then the hair is shampooed and rinsed to complete the hair dyeing process.

In the hair dyeing method of the present invention adopting the disulfide bond reduction, the color of the product can be a mixed ointment of a one-dose color and a disulfide bond reducer, or two different ointments of two doses (such as a first-dose dye ointment which is the color, and a second-dose ointment which is the disulfide bond reducer), and the two doses of ointments are mixed with each other and prepared before their use for dyeing hair, and an alkali may or may not be added into this dose depending on the properties of the disulfide bond reducer. The one-dose ointment can be used directly without adding the alkali, and the hair dyeing procedure is as follows.
1. After the ointment of this hair dye is coated uniformly on each hair by a hair dyeing brush, the ointment stays on the hair for 20-30 minutes, and a hair dryer or a steamer can be used for heating the hair during the stay period.
2. This ointment is washed away by warm water, and then the hair is shampooed and rinsed to complete the hair dyeing process.

The colors used in this hair dyeing method of the present invention include all natural colors (used in food and cosmetics), synthetic colors (used in food and cosmetics), and cosmetic colors, and the hair dye is the permanent hair dye. Wherein, the disulfide bond reducer can be D, L-cysteine and its salts or a derivative of cysteine (N-acetyl-D, L-cysteine), thiol acid or their salts and esters, thiolactic acid and its salts, sulfurous acid and its salts, ascorbic acid and its salts, L-ascorbic acid (vitamin C) and its salts, and the aforementioned compounds. In addition, a hair penetration enhancer and a hair expander can be added to the aforementioned compounds to improve the hair dyeing performance, wherein the expander and the penetration enhancer can be benzyl alcohol, isopropanol, N-methyl-2-pyrolidone, propylene carbonate, sodium sulphate anhydrous, benzoic acid, formic acid, urea, guanidine, sodium dodecyl sulfate, sodium lauryl sarcosinate, Triton X-100, 1-dodecylazacycloheptan-2-one, Persons skilled in the art should know that any equivalent reagent can be used instead .

### (3) Metal Mordant Dyeing Method

The hair dyeing method adopting the metal mordant dyeing product of the present invention can be divided into three-dose and two-dose products, and the first dose of the three-dose product is a disulfide bond reducer ointment and this dose is provided for breaking the disulfide bond to open the cuticle to facilitate color molecules of the next dose to enter into the hair cortex layer. However, if the dyeing hair is black hair or any other dark hair, then this dose can be changed to the so-called bleach (which is a black hair discoloration agent) and the bleach is provided for lightening the black color of the hair into a yellow to brown hair color, as well as breaking the disulfide bonds to open the cuticle, and the aforementioned two effects can help the metal ions of the next dose to enter into the cortex layer successfully. The second dose is a low-toxicity metal ion ointment, and the third dose is a color dye ointment. When hair is dyed, the first dose, second dose and third dose ointment are used according to a specific sequence, and the first dose of the two-dose product is a mixed ointment of metal ions and a disulfide bond reducer, wherein the metal ions and the disulfide bond reducer can be separate ointments, and these two ointments are mixed directly before use. The alkali is added depending on the type of the disulfide bond reducer, and the ointments of the first dose and the second dose ointment are used according to a specific sequence for dyeing hair, and the hair dyeing procedure is described as follows.
(I) Hair Dyeing Procedure of Three-Dose Hair Dyes
1. A hair dyeing brush is used to apply the first dose (disulfide bond reducer ointment) onto each hair uniformly, and then the first dose stays on the hair for 10 minutes directly, and the hair is heated by a hair dryer or a steamer during the stay period, and then the ointment on the hair is washed away by warm water, or the next step is carried out directly without washing away the ointment. If the dyeing hair is black hair or any dark hair, then this dose can be changed to 6% peroxide cream plus bleach (black hair discoloration agent), and the bleach stays on the hair for 30 minutes, and then it is necessary to wash away the bleach.
2. A hair dyeing brush is used to apply the second dose (metal ion ointment) onto each hair uniformly, and the second dose stays on the hair for 15 minutes directly, and the hair is heated by a hair dryer or a steamer during the stay period, and then this ointment on the hair is washed away by warm water.
3. A hair dyeing brush is used to apply the third dose (color dye ointment) onto each hair uniformly, and the third dose stays on the hair for 15 minutes directly, and the hair can be heated by a hair dryer or a steamer during the stay period, and then this ointment on the hair is washed away by warm water and then the hair is shampooed and rinsed to complete the hair dyeing process.
(II) Hair Dyeing Procedure of Two-Dose Hair Dyes
1. A hair dyeing brush is used to apply the first dose (metal ion ointment) onto each hair uniformly, and the first dose stays on the hair for 15 minutes directly, and the hair is heated by a hair dryer or a steamer during the stay period, and then this ointment on the hair is washed by warm water.
2. A hair dyeing brush is used to coat the second dose (color dye ointment) onto each hair uniformly, and the second dose stays on the hair for 15 minutes directly, and the hair is heated by a hair dryer or a steamer during the stay period, and then this ointment on the hair is washed by warm water, and then the hair is shampooed and rinsed to complete the hair dyeing process.

The colors applicable for the aforementioned hair dyeing method of the present invention include natural colors (used in food and cosmetics) of anions, cations, NH₂-based, OH-based, and SH-based ligands, synthetic colors (used in food and cosmetics) and cosmetic colors, and the hair dye of this sort is categorized as the permanent hair dye, and its hair dyeing results are listed in Table 8, wherein the disulfide bond reducer is the same as the disulfide bond reduction described in the aforementioned embodiments, and the metal ions include low-toxicity magnesium, aluminum, iron, and zinc salts, and persons skilled in the art should know that any equivalent reagent can be used instead.

TABLE 8
List of hair dyeing results of metal mordant dyeing

| | | potassium | | | | | |
|---|---|---|---|---|---|---|---|
| | aluminum | aluminum | magnesium | magnesium | zinc | ferric | ferrous |
| Colors/Metal | sulfate | sulfate | sulfate | chloride | chloride | chloride | sulfate |
| Ion | Formed Color | | | | | | |
| Purple cabbage | Blue | Blue | purple red | purple red | purple | purple | purple |
| colors | | | | | red | Blue | Blue |
| Annatto yellow | yellow | yellow | yellow | yellow | yellow | yellow | yellow |
| colors | orange | orange | orange | orange | orange | orange | orange |
| Gardenia | orange | orange | orange | orange | orange | orange | orange |
| yellow colors | | | | | | | |
| Tumeric yellow | Yellow | Yellow | Yellow | Yellow | Yellow | Yellowish | Yellowish |
| colors | | | | | | Brown | Brown |
| Grape skin | Bluish | Bluish | Bluish | Bluish | Bluish | Bluish | Bluish |
| colors | Purple | Purple | Purple | Purple | Purple | Purple | Purple |
| Monascuscolors | Dark Red | Dark Red | Dark Red | Dark Red | Dark | Dark Red | Dark Red |
| | | | | | Red | | |
| Chlorophyll | Fresh | Fresh | Fresh | Fresh | Fresh | Fresh | Fresh |
| | Green | Green | Green | Green | Green | Green | Green |
| Carmine colors | Purplish | Purplish | Purplish | Purplish | Purplish | Purplish | Purplish |
| | Red | Red | Red | Red | Red | Red | Red |
| Beetroot colors | Pink | Pink | Pink | Pink | Pink | Pink | Pink |

### (4) Ionic Bond Method

In the hair dyeing method using the ionic bond method of the present invention, the product is in a one-dosage form, and its composition includes an anion or cation color which is primarily a dye ointment, and an hair expander and a penetration enhancer can be added into the ointment to improve the hair dyeing effect, and an acid can be added to the color containing anions to improve the hair dyeing effect, and an alkali can be added to the color containing cations to improve the hair dyeing effect, or a hair dryer or a steamer is used for heating the hair to improve the hair dyeing effect by a physical method, and this method is the same as the hair dyeing method of a semi-permanent hair dye available in the market, and its hair dyeing procedure is described below.
1. The dye ointment is coated uniformly onto each hair by a hair dyeing brush and stays on the hair for 20-30 minutes, and then the dye on the hair is washed by warm water, and a hair dryer or a steamer can be used during the stay period to heat the hair;
2. The ointment on the hair is washed by warm water, and then the hair is shampooed and rinsed to complete the hair dyeing process.

The colors applicable for this hair dyeing method of the present invention include natural colors (used in food and cosmetics), synthetic colors (used in food and cosmetics), and cosmetic colors, containing anions or cations, and the hair dye of this sort is categorized as the semi-permanent hair dye, wherein the acid refers to an organic acid or an inorganic acid, and the alkali refers to an organic alkali or an inorganic alkali, which are the same as those used in the hair oxidation method of the foregoing preferred embodiment, and the expander and penetration enhancer are the same as those used in the disulfide bond reduction of the foregoing preferred embodiments, and persons skilled in the art should know that any equivalent reagent can be used instead .

### (5) Adsorption Method

In the hair dyeing method using the adsorption method of the present invention, the product is in a dosage form, and the color is primarily a dye ointment, and this method is the same as the hair dyeing method of a temporary hair dye available in the market, and its hair dyeing procedure is described as follows.
1. A hair dyeing brush is used to coat a dye ointment onto each hair uniformly.
2. A hair dryer is used to blow dry the hair to complete the hair dyeing process.

The colors applicable for this hair dyeing method of the present invention include natural colors (used in food and cosmetics), synthetic colors (used in food and cosmetics) and cosmetic colors, and the hair dye of this sort is categorized as the temporary hair dye.
(III) The dyeing and perming products using natural colors (used in food and cosmetics), synthetic colors (used in food and cosmetics) and cosmetic colors are described in details as follows.

In a general perm, a two-dose and two-step procedure is usually taken, and the first dose is mainly composed of two types of disulfide bond reducers (such as the disulfide bond reducer used in the disulfide bond reduction), such as thiol acid or its salts and esters or cysteine, and the second dose is mainly composed of two types of disulfide bond oxidizers, such as bromate or hydrogen peroxide. The basic perming principle is to use the first-dose disulfide bond reducer to break the disulfide bonds between hair proteins to form —SH base, and then use the second-dose disulfide bond oxidizer to rebuild the —SH base to form new disulfide bonds, so as to achieve the effect of shaping hair. At present, the perming procedure and dyeing procedure are performed separately, and it is necessary to reserve a week or more for the two procedures, and each procedure takes at least one hour, and thus it will save much time for individuals or hair stylists and simplify the complicated dyeing and perming procedures, if the two procedures can be combined into one and completed all at a time.

As described above, the primary objective of the present invention is to combine the dyeing and perming processes, such that the design requires a mixture of dyeing and perming materials that will not affect their functions by each other. According to the principle of a perm, the first-dose disulfide bond reducer is mainly provided for breaking the disulfide bonds of hair proteins, while opening the hair cuticle, and this principle is the same as the hair dyeing principle by the disulfide bond reduction, and thus the first design method of this dyeing and perming product is achieved by the aforementioned disulfide bond reduction of the hair, wherein the disulfide bond reduction type hair dye ointment is used as a first dose of the dyeing and perming product and this dose is provided for breaking the disulfide bonds of the hair and promoting the color molecules to enter and deposit into the cortex layer of the hair, and then a disulfide bond oxidizer is used as a second dose of the dyeing and perming product, and this dose is provided for rebuilding the disulfide bond to achieve the effect of shaping the hair and oxidizing and discoloring the colors that are not aligned with the cortex layer, so as to achieve the dyeing and perming effects, and a product of this sort is categorized as a two-dose dyeing and perming product.

The second design method of the dyeing and perming product is achieved by the oxidation of the aforementioned hair dye. Similarly, the first-dose disulfide bond reducer is used for breaking the disulfide bonds of the hair protein and opening the hair cuticles, and then a mixed ointment of the color ointment and hydrogen peroxide or peroxide ointment (which is a disulfide bond oxidizer) used in the aforementioned oxidation type hair dyeing method is used as the second dose for rebuilding the disulfide bond by the oxidation by hydrogen peroxide or peroxide to achieve the effects of shaping the hair and promoting color molecules to enter and deposit into the cortex layer of the hair, so as to achieve the dyeing and perming effects, and a product of this sort is categorized as the two-dose dyeing and perming product. The third design method of the dyeing and perming product does not mix the color ointment with the hydrogen peroxide or peroxide ointment (which is a disulfide bond oxidizer), and the hair dyeing and perming are performed separately. Similarly, the first-dose disulfide bond reducer is used for breaking the disulfide bonds of the hair protein, and then a second-dose color ointment is used for dyeing hair, and finally a third-dose disulfide bond oxidizer is used for rebuilding the disulfide bonds and achieving the effect of shaping hair, and a product of this sort is categorized as a three-dose dyeing and perming product. In the fourth design method of the dyeing and perming product, the disulfide bond reducer and the color are designed with the same ointment and used as a first dose for breaking the disulfide bond of the hair protein, and opening the hair cuticle, and then oxygen generated from air or by an oxygen generator is used as a second dose (which is a disulfide bond oxidizer), and the oxygen oxidizes and rebuilds the disulfide bonds to achieve the effects of shaping hair and promoting color molecules to enter and deposit into the hair cortex layer, so as to achieve the dyeing and perming effects, and a product of this sort is categorized as a two-dose dyeing and perming product.

There are three kinds of perming methods, respectively: a thermoplastic method, a lotion wrap method, and a cold perming method, and the operating procedure of the dyeing and perming product using these three perming methods are described in details as follows.

### 1. Thermoplastic Method: Operating Procedure of Two-Dose Dyeing and Perming Products

Step 1: Shampoo (in a wet condition).
Step 2: Apply a first dose (remain for 20 minutes).
Step 3: Rinse.
Step 4: Set rollers.
Step 5: Heat, set, and dry (fully dry).
Step 6: Apply a second dose (remain for 20 minutes).
Step 7: Remove rollers.
Step 8: Rinse.

### 2. Lotion Wrap Method: Operating Procedure of Two-Dose Dyeing and Perming Products

Step 1: Shampoo (in a wet condition).
Step 2: Apply a first dose (remain for 20 minutes).
Step 3: Set rollers.
Step 4: Heat, set, and dry (fully dry).
Step 5: Apply a second dose (remain for 20 minutes).
Step 6: Remove rollers.
Step 7: Rinse.

### 3. Cold Perming Method: Operating Procedure of Two-Dose Dyeing and Perming Products

Step 1: Shampoo (in a wet condition).
Step 2: Apply a first dose (remain for 20 minutes).
Step 3: Rinse.
Step 4: Set rollers.
Step 5: Apply a second dose (remain for 20 minutes).
Step 6: Remove rollers.
Step 7: Rinse.

### 4. Thermoplastic Method: Operating Procedure of Three-Dose Dyeing and Perming Products

Step 1: Shampoo (in a wet condition).
Step 2: Apply a first dose (remain for 10 minutes).
Step 3: Apply a second dose (remain for 20 minutes).
Step 4: Rinse.
Step 5: Set rollers.
Step 6: Heat, set, and dry (fully dry).
Step 7: Apply a third dose (remain for 10 minutes).
Step 8: Remove rollers.
Step 9: Rinse

### 5. Lotion Wrap Method: Operating Procedure of Three-Dose Dyeing and Perming Products

Step 1: Shampoo (in a wet condition).
Step 2: Apply a first dose (remain for 10 minutes).
Step 3: Apply a second dose (remain for 20 minutes).
Step 4: Set rollers.
Step 5: Heat, set, and dry (fully dry).
Step 6: Apply a second dose (remain for 10 minutes).
Step 7: Remove rollers.
Step 8: Rinse.

### 6. Cold Perming Method: Operating Procedure of Three-Dose Dyeing and Perming Products

Step 1: Shampoo (in a wet condition).
Step 2: Apply a first dose (remain for 10 minutes).
Step 3: Apply a second dose (remain for 20 minutes).
Step 4: Rinse.
Step 5: Set rollers.
Step 6: Apply a third dose (remain for 10 minutes).
Step 7: Remove rollers.
Step 8: Rinse

The source of colors applicable for this dyeing and perming product of the present invention include natural colors (used in food and cosmetics), synthetic colors (used in food and cosmetics), and cosmetic colors; and the color has a content of 1% to 10% by weight and an appropriate content of 2% to 5%, wherein the disulfide bond reducer is the same as that used in the disulfide bond reduction of the aforementioned preferred embodiment, and the disulfide bond reducer has a content of 1% to 10% by weight and an appropriate content of 3% to 8%. If it is necessary to adjust the pH value of the disulfide bond reducer, then the alkali is adjusted, and the consumption of the alkali is up to a level primarily adjusting the pH value of the ointment of the product ointment to 7.5-9.5, and the actual consumption depends on the properties of the alkali, wherein the alkali can be the same organic alkali or inorganic alkali as those used in the disulfide bond reduction of the aforementioned preferred embodiments, and the disulfide bond reducer/oxidizer can be made of the same material as the second dose of the perming product such as hydrogen peroxide and bromate, or the same organic peroxide and inorganic peroxide, or reagent or kit that generates oxygen used in the oxidation of the aforementioned preferred embodiment, and the disulfide bond reducer/oxidizer has a content of 3% to 12% by weight and an appropriate content of 3% to 8%. If it is necessary to adjust the pH value of the disulfide bond oxidizer, then an acid is used for the adjustment, and the consumption of acid is primarily up to a level for adjusting the pH value of the ointment of the product ointment to 2.5-4.5, and the actual consumption depends on the properties of the acid, wherein the acid refers to an organic acid or an inorganic acid. If it is necessary to adjust the pH value of the ointment in the color, an organic acid, an inorganic acid, an organic alkali or an inorganic alkali can be added for the adjustment, and persons skilled in the art should know that any equivalent reagent can be used instead. The dosage of the present invention can be in form of a solution, lotion, cream, frost, gel, paste, foam or spray.
(IV) The color of the hair dye produced by mixing color dyes and the hair dyeing method using this color are described in details.

The colors of the dye in the present invention primarily include red, yellow, blue, pink, green, orange, tangerine, purple, black, and white, and any required dye color can be prepared by mixing the colors according to the principle of mixing the drawing paints. The present invention uses a food color dye with five single colors including Taiwan Food Yellow No. 5 (FD&C Yellow No. 6), Taiwan Food Yellow No. 4 (FD&C Yellow No. 5), Taiwan Food Red No. 6 (Acid Red 18), Taiwan Food Red No. 7 (FD&C Red No. 3), and Taiwan Food Blue No. 1 (FD&C Blue No. 1) used for preparing 106 kinds of color dye, and the strictest oxidation hair dyeing experiment is performed to confirm the dyeing effect on black hair, and experiment results show that the way of preparing the dye by mixing different colors can dye the colors onto black hair successfully, and the results of the wash persistency and durability of this dye obtained from the strictest experiment taken after 30 times of shampoo of the dyed hair as shown in Table 9 show that this dye is a permanent hair dye.

TABLE 9
Results of dyes prepared by mixing different colors according to
percentage by weight

| New No. | Y4 | Y5 | R6 | R7 | B1 | Pantone No. |
|---|---|---|---|---|---|---|
| 1 | 0.8 | 0.2 | 0.0 | 0.0 | 0.0 | 1235c |
| 2 | 0.6 | 0.4 | 0.0 | 0.0 | 0.0 | 1375c |
| 3 | 0.4 | 0.6 | 0.0 | 0.0 | 0.0 | 1375c |
| 4 | 0.2 | 0.8 | 0.0 | 0.0 | 0.0 | 1375c |
| 5 | 0.2 | 0.6 | 0.2 | 0.0 | 0.0 | 1585c |
| 6 | 0.4 | 0.4 | 0.2 | 0.0 | 0.0 | 1585c |
| 7 | 0.0 | 0.8 | 0.2 | 0.0 | 0.0 | 1505c |
| 8 | 0.6 | 0.2 | 0.2 | 0.0 | 0.0 | 1505c |
| 9 | 0.8 | 0.0 | 0.2 | 0.0 | 0.0 | 1505c |
| 10 | 0.2 | 0.4 | 0.0 | 0.4 | 0.0 | 1787c |
| 11 | 0.4 | 0.0 | 0.4 | 0.2 | 0.0 | 1787c |
| 12 | 0.2 | 0.2 | 0.4 | 0.2 | 0.0 | 1787c |
| 13 | 0.0 | 0.6 | 0.0 | 0.4 | 0.0 | 1789c |
| 14 | 0.6 | 0.0 | 0.4 | 0.0 | 0.0 | 1788c |
| 15 | 0.4 | 0.4 | 0.0 | 0.2 | 0.0 | 1787c |
| 16 | 0.4 | 0.2 | 0.2 | 0.2 | 0.0 | 1787c |
| 17 | 0.0 | 0.4 | 0.6 | 0.0 | 0.0 | 1787c |
| 18 | 0.0 | 0.6 | 0.4 | 0.0 | 0.0 | 1787c |
| 19 | 0.2 | 0.2 | 0.6 | 0.0 | 0.0 | 1787c |
| 20 | 0.2 | 0.4 | 0.4 | 0.0 | 0.0 | 1787c |
| 21 | 0.8 | 0.0 | 0.0 | 0.2 | 0.0 | 1787c |
| 22 | 0.2 | 0.6 | 0.0 | 0.2 | 0.0 | 1787c |
| 23 | 0.2 | 0.0 | 0.8 | 0.0 | 0.0 | 1787c |
| 24 | 0.4 | 0.2 | 0.4 | 0.0 | 0.0 | 1787c |
| 25 | 0.4 | 0.0 | 0.6 | 0.0 | 0.0 | 1787c |
| 26 | 0.2 | 0.4 | 0.2 | 0.2 | 0.0 | 1787c |
| 27 | 0.6 | 0.0 | 0.2 | 0.2 | 0.0 | 1787c |
| 28 | 0.2 | 0.2 | 0.2 | 0.4 | 0.0 | 1787c |
| 29 | 0.6 | 0.2 | 0.0 | 0.2 | 0.0 | 1787c |
| 30 | 0.0 | 0.6 | 0.2 | 0.2 | 0.0 | Red c |
| 31 | 0.0 | 0.8 | 0.0 | 0.2 | 0.0 | Red c |
| 32 | 0.4 | 0.0 | 0.0 | 0.6 | 0.0 | Red032c |
| 33 | 0.6 | 0.0 | 0.0 | 0.4 | 0.0 | Red032c |
| 34 | 0.0 | 0.2 | 0.2 | 0.6 | 0.0 | Red032c |
| 35 | 0.4 | 0.2 | 0.0 | 0.4 | 0.0 | Red032c |
| 36 | 0.0 | 0.4 | 0.2 | 0.4 | 0.0 | 185c |
| 37 | 0.0 | 0.2 | 0.6 | 0.2 | 0.0 | 185c |
| 38 | 0.0 | 0.2 | 0.4 | 0.4 | 0.0 | 185c |
| 39 | 0.0 | 0.0 | 0.8 | 0.2 | 0.0 | 185c |
| 40 | 0.0 | 0.2 | 0.8 | 0.0 | 0.0 | 185c |
| 41 | 0.0 | 0.4 | 0.4 | 0.2 | 0.0 | 1925c |
| 42 | 0.0 | 0.0 | 0.6 | 0.4 | 0.0 | 1925c |
| 43 | 0.0 | 0.4 | 0.0 | 0.6 | 0.0 | 1925c |
| 44 | 0.0 | 0.2 | 0.0 | 0.8 | 0.0 | 1925c |
| 45 | 0.2 | 0.2 | 0.0 | 0.6 | 0.0 | 219c |
| 46 | 0.0 | 0.0 | 0.4 | 0.6 | 0.0 | 219c |
| 47 | 0.2 | 0.0 | 0.0 | 0.8 | 0.0 | 219c |
| 48 | 0.0 | 0.0 | 0.2 | 0.8 | 0.0 | 219c |
| 49 | 0.0 | 0.4 | 0.0 | 0.0 | 0.6 | 3308c |
| 50 | 0.2 | 0.4 | 0.0 | 0.0 | 0.4 | 343c |
| 51 | 0.4 | 0.2 | 0.0 | 0.0 | 0.4 | 348c |
| 52 | 0.4 | 0.0 | 0.0 | 0.0 | 0.6 | 348c |
| 53 | 0.2 | 0.2 | 0.0 | 0.0 | 0.6 | 349c |
| 54 | 0.0 | 0.6 | 0.0 | 0.0 | 0.4 | 349c |
| 55 | 0.6 | 0.2 | 0.0 | 0.0 | 0.2 | 349c |
| 56 | 0.4 | 0.4 | 0.0 | 0.0 | 0.2 | 349c |
| 57 | 0.8 | 0.0 | 0.0 | 0.0 | 0.2 | 355c |
| 58 | 0.6 | 0.0 | 0.0 | 0.0 | 0.4 | 356c |
| 59 | 0.0 | 0.8 | 0.0 | 0.0 | 0.2 | 371c |
| 60 | 0.2 | 0.6 | 0.0 | 0.0 | 0.2 | 378c |
| 61 | 0.2 | 0.0 | 0.0 | 0.0 | 0.8 | 569c |
| 62 | 0.0 | 0.2 | 0.0 | 0.0 | 0.8 | 7473c |
| 63 | 0.0 | 0.2 | 0.2 | 0.0 | 0.6 | 432c |
| 64 | 0.0 | 0.0 | 0.4 | 0.0 | 0.6 | 7463c |
| 65 | 0.0 | 0.0 | 0.2 | 0.0 | 0.8 | 7546c |
| 66 | 0.0 | 0.0 | 0.0 | 0.2 | 0.8 | 526c |
| 67 | 0.0 | 0.0 | 0.0 | 0.4 | 0.6 | 525c |
| 68 | 0.0 | 0.0 | 0.2 | 0.2 | 0.6 | 5185c |
| 69 | 0.0 | 0.2 | 0.0 | 0.2 | 0.6 | 515c |
| 70 | 0.0 | 0.0 | 0.8 | 0.0 | 0.2 | 5125c |
| 71 | 0.0 | 0.0 | 0.6 | 0.0 | 0.4 | 438c |
| 72 | 0.0 | 0.0 | 0.4 | 0.2 | 0.4 | 262c |
| 73 | 0.0 | 0.2 | 0.0 | 0.4 | 0.4 | 262c |
| 74 | 0.2 | 0.2 | 0.0 | 0.2 | 0.4 | 261c |
| 75 | 0.0 | 0.0 | 0.2 | 0.4 | 0.4 | 261c |
| 76 | 0.0 | 0.0 | 0.0 | 0.6 | 0.4 | 229c |
| 77 | 0.0 | 0.2 | 0.0 | 0.6 | 0.2 | 229c |
| 78 | 0.0 | 0.6 | 0.0 | 0.2 | 0.2 | 229c |
| 79 | 0.0 | 0.4 | 0.0 | 0.2 | 0.4 | 228c |
| 80 | 0.0 | 0.0 | 0.6 | 0.2 | 0.2 | 228c |
| 81 | 0.6 | 0.0 | 0.0 | 0.2 | 0.2 | 228c |
| 82 | 0.0 | 0.0 | 0.0 | 0.8 | 0.2 | 227c |
| 83 | 0.0 | 0.0 | 0.2 | 0.6 | 0.2 | 227c |
| 84 | 0.4 | 0.0 | 0.0 | 0.4 | 0.2 | 227c |
| 85 | 0.0 | 0.0 | 0.4 | 0.4 | 0.2 | 227c |
| 86 | 0.4 | 0.0 | 0.2 | 0.2 | 0.2 | 222c |
| 87 | 0.2 | 0.2 | 0.0 | 0.4 | 0.2 | 221c |
| 88 | 0.4 | 0.2 | 0.0 | 0.2 | 0.2 | 215c |
| 89 | 0.0 | 0.4 | 0.0 | 0.4 | 0.2 | 215c |
| 90 | 0.2 | 0.4 | 0.0 | 0.2 | 0.2 | 215c |
| 91 | 0.2 | 0.2 | 0.4 | 0.0 | 0.2 | 440c |
| 92 | 0.2 | 0.4 | 0.2 | 0.0 | 0.2 | 450c |
| 93 | 0.0 | 0.6 | 0.2 | 0.0 | 0.2 | 449c |
| 94 | 0.0 | 0.4 | 0.4 | 0.0 | 0.2 | 491c |
| 95 | 0.0 | 0.2 | 0.6 | 0.0 | 0.2 | 492c |
| 96 | 0.4 | 0.0 | 0.4 | 0.0 | 0.2 | 498c |
| 97 | 0.0 | 0.2 | 0.4 | 0.0 | 0.4 | 5195c |
| 98 | 0.0 | 0.4 | 0.2 | 0.0 | 0.4 | 532c |
| 99 | 0.2 | 0.2 | 0.2 | 0.0 | 0.4 | 5467c |
| 100 | 0.6 | 0.0 | 0.2 | 0.0 | 0.2 | 553c |
| 101 | 0.4 | 0.2 | 0.2 | 0.0 | 0.2 | 5605c |
| 102 | 1 | 0 | 0 | 0 | 0 | Yellow C |
| 103 | 0 | 1 | 0 | 0 | 0 | Orange 21C |
| 104 | 0 | 0 | 1 | 0 | 0 | Red 32 |
| 105 | 0 | 0 | 0 | 1 | 0 | Rhodamine Red C |
| 106 | 0 | 0 | 0 | 0 | 1 | Blue 072C |

(V) The hair dye that changes colors with different pH values is described in details as follows.

Among the hair dyes disclosed in the present invention, there is a natural food color that can changes the color of the dye with the pH value, such as the natural color containing anthocyanin, wherein anthocyanin has the color changing effect similar to a pH indicator, and its color varies with pH value. If the pH value is increased gradually from 1 to 13, the color will change from red , bluish red, purple, blue, green, and then yellow, and thus this feature can be used for developing a color changing hair dye. Table 10 roughly lists the natural colors that can change colors. In conjunction with the aforementioned hair dyeing methods such as the metal mordant dyeing, disulfide bond reduction, oxidation, ionic bond method and adsorption methods. The natural colors listed in Table 10 can penetrate the natural color into target positions (such as the cuticle or hair cortex) of the hair by using different hair dyeing methods according to different requirements to produce a permanent, semi-permanent, or temporary hair. The natural color monomer capable of changing color with pH value in accordance with the present invention includes anthocyanin, carminic acid, betanin, vulgaxanthin, santalin, bixin, and norbixin, and plant colors and extracts having molecules of this color, wherein the color changing hair dye can be formed by a single-color dye or a dye produced by mixing different colors in any proportion, and hair can be dyed according to different hair dyeing methods, and buffer solutions of different pH values are sprayed onto the hair, and the hair is blown dry by a hair dryer to complete the color change of the hair. In the description above, the natural colors capable of changing color with pH value are covered by the scope of the present invention, and the content of color in the color ointment is 1% to 10% by weight and the appropriate content is 2% to 5% by weight.

The pH buffer refers to an acid, a base, a salt and its compound or buffer solution, and the acid can be an organic acid, an inorganic acid, or its salts, and the base can be an organic base, aninorganic base alkali and its salts, and the buffer solution has a pH value of 2.0-10.0 and any equivalent. The hair dye of the present invention can be manufactured in form of a solution, lotion, cream, frost, gel, paste, foam, spray or shampoo.

TABLE 10
Types of natural colors capable of changing color with pH value

| | | Main composition of the | Color Change |
|---|---|---|---|
| No. | Colors | colors | Range |
| 1 | Grape skin | anthocyanin | Red to purple |
| 2 | Aronia berry | anthocyanin | Red to purple |
| 3 | Black gooseberry | anthocyanin | Red to purple |
| 4 | Black carrot | anthocyanin | Red to purple |
| 5 | Elderberry | anthocyanin | Red to purple |
| 6 | Roselle | anthocyanin | Red to purple |
| 7 | Beetroot | betanin | Blue to purple |
| | | vulgaxanthin | |
| 8 | Carmine | carminic acid | Red to orange |
| 9 | Sandalwood | santalin | Orange to red |
| 10 | Annatto | Bixin (oil-soluble) | Yellow to orange |
| | | Norbixin(water-soluble) | |

(VI) Optimal Conditions of Hair Dyes

### (1) Optimal Conditions of Oxidation Permanent Hair Dye Products

This hair dye can be divided into three-dose and two-dose products, and the three-dose product includes a first-dose color dye ointment, a second-dose hydrogen peroxide or peroxide ointment, a third-dose alkali ointment or powder; and the two-dose product contains a first dose that combines a color and an alkali, and the second dose is still the hydrogen peroxide or peroxide ointment. All ointments are mixed before use. The sources of colors include natural colors (used in food and cosmetics), synthetic colors (used in food and cosmetics), and cosmetic colors. The color ointment has a content of 1% to 10% by weight percentage, and an appropriate content of 2% to 5%. The hydrogen peroxide, peroxide or their compound in the ointment has a content of 3% to 12% by weight and an appropriate content of 3% to 6%; and the consumption of the alkali ointment or powder is up to a level sufficient to adjust the pH value of the ointment in the product to 7.5-8.5, and the actual consumption depends on the properties of the alkali. The alkali can be an organic alkali or an inorganic alkali, and practical examples of the organic alkali include alkylamines such as monoethanolamine, diethanolamine, triethanolamine, monopropanolamine, dipropanolamine, tripropanolamine, 2-amino-2-methyl-1,3-propanediol, 2-amino-2-methyl- l-propanol and 2-amino-2-hydroxmethyl -1,3-propanediol; an inorganic salt such as sodium hydroxide, sodium carbonate, sodium bicarbonate, calcium carbonate and calcium bicarbonate; and a guanidine salt such as guanidine hydrochloride, guanidine sulfate, guanidine nitrate, guanidine phosphate, guanidine phosphate sulfate, guanidine borate, aminoguanidine sulfate, and a guanidine salt of an organic acid. The hair dye of the present invention can be manufactured in form of a solution, lotion, cream, frost, gel, paste, foam, spray, or shampoo.

### (2) Optimal Conditions of Permanent Hair Dye Products Using Disulfide Bond Reduction

This hair dye can be divided into two-dose and one-dose product, wherein the first dose of the two-dose product is an ointment which is a disulfide bond reducer ointment, and the second dose is the dye ointment, and the first-dose and second-dose ointments are applied according to a sequence or the two doses are mixed directly before use. If it is necessary to add an alkali, the alkali is combined with the ointment (or disulfide bond reducer) and applied directly to the hair for dyeing the hair. The one-dose product is an ointment containing a disulfide bond reducer, a color and an alkali as a whole, so that it can be applied directly. The sources of colors include natural colors (used in food and cosmetics), synthetic colors (used in food and cosmetics) and cosmetic colors; the color in the ointment has a content of 1% to 10% by weight and an appropriate content of 2% to 5%; the disulfide bond reducer in the ointment has a content of 1% to 10% by weight and an appropriate content of 3% to 8%, wherein the disulfide bond reducer can be D, L-cysteine and its salts, a derivative of cysteine (N-acetyl-D, L-cysteine), thiol acid or its salts and esters, thiolactic acid and its salts, sulfurous acid and its salts, ascorbic acid and its salts, L-ascorbic acid (vitamin C) and its salts, or any equivalents. Persons skilled in the art should know that any equivalent reagent can be used instead. The consumption of the alkali ointment or powder is up to a level sufficient to adjust the pH value of the ointment in the product to 8.0-9.0 and the actual consumption depends on the properties of the alkali. The alkalis are the same as those described above.

The hair dye of the present invention can be manufactured in form of a solution, lotion, cream, frost, gel, paste, foam, spray, or shampoo.

(3) Optimal Conditions of Permanent Hair Dye Products Using Metal Mordant Dyeing Method

This hair dye can be divided into a three-dose product and a two-dose product, wherein the first dose of the three-dose product is an ointment which is a disulfide bond reducer provided for breaking the disulfide bond to open the cuticle and facilitate color molecules of the next dose to enter into the cortex layer of the hair. If the dyeing hair is the black hair or other dark hair, then this dose is substituted by the so-called “bleach” (black hair discoloration agent). One of the purposes of the bleach is to lighten the black hair color of the hair to a yellow to brown hair color, and the other purpose of the bleach is to break the disulfide bond to open the cuticle and facilitate metal ions of the next dose to enter into the cortex layer. The second dose is a low-toxicity metal ion ointment, and the third dose is a color dye ointment. The first-dose, second-dose, and third-dose ointments are applied in sequence for dyeing hair. The first dose of the two-dose product is a mixed ointment containing metal ions and a disulfide bond reducer, and the metal ions and the disulfide bond reducer can be separate ointments, and these two doses of ointments are mixed directly before use, and the alkali is added depending on the type of the disulfide bond reducer, and the second dose is a color dye ointment, and the first-dose and second-dose ointments are applied in sequence for dyeing hair. The source of colors includes natural colors (used in food and cosmetics), synthetic colors (used in food and cosmetics), and cosmetic colors; and the color ointment has a content of 1% to 10% by weight and an appropriate content of 2% to 5%. The metal ion refers to a low-toxicity magnesium, aluminum, iron, or zinc salt or any equivalent, and the metal ions have a content of 1% to 10% by weight and an appropriate content of 2% to 5%. The disulfide bond reducer in the disulfide bond reducer ointment has a content of 1% to 10%, by weight and an appropriate content of 3% to 8%, wherein the disulfide bond reducer is the same as those described above, and the alkali is the same as those described above. The hair dye of the present invention can be manufactured in form of a solution, lotion, cream, frost, gel, paste, foam, spray or shampoo.

### (4) Optimal Condition of Semi-permanent Hair Dye Products Using Ionic Bond Method

This hair dye is a one-dose product comprising anion or cation colors, and the ointment also contains a hair expander and a penetration enhancer. In addition, an acid can be added to the color containing anions to improve the hair dyeing effect, and an alkali can be added to the color containing cations to improve the hair dyeing effect, and the hair can be heated by a hair dryer or a steamer to improve the hair dyeing effect by a physical method, and all these can be used directly for dyeing hair. The source of colors includes natural colors (used in food and cosmetics), synthetic colors (used in food and cosmetics) and cosmetic colors containing anions or cations, and the color in the color ointment has a content of 1% to 10% by weight percentage and an appropriate content of 2% to 5%. The acid can be an organic acid or an inorganic acid, and its consumption depends on the properties of the acid and primarily aims at adjusting the pH value of the ointment to 2.0-6.0, and includes hydrochloric acid, tartaric acid, citric acid, phosphoric acid and any equivalent. The alkali can be an organic alkali or an inorganic alkali, and the types of alkalis are the same as those described above, and its consumption depends on the properties of the alkali, and primarily aims at adjusting the pH value of the ointment to 8.0-10.0. The expander and penetration enhancer are the same as those described above, and have a content of 3% to 20% by weight and an appropriate content of 5% to 15%, and persons skilled in the art should know that any equivalent reagent can be used instead. The hair dye of the present invention can be manufactured in form of a solution, lotion, cream, frost, gel, paste, foam, spray or shampoo.

### (5) Optimal Conditions of Adsorption Temporary Hair Dye Product

This hair dye is a one-dose product comprising color ointments and can be used directly for dyeing hair. The source of colors includes natural colors (used in food and cosmetics), synthetic colors (used in food and cosmetics) and cosmetic colors, and the color in the color ointment has a content of 1% to 10% by weight and an appropriate content of 2% to 5%. The hair dye of the present invention can be manufactured in form of a solution, lotion, cream, frost, gel, paste, foam, spray or shampoo.

In addition the description above, other general compositions used in hair dyes can be adopted to meet the same requirements without sacrificing the effect of the present invention, and the composition can be a cation surfactant, anion surfactant, non-ionic surfactant, amphoteric surfactant, lanolin derivative, protein derivative, oil, fat, wax, hydrocarbon, polyhydroxy alcohol, ester, amide, moisturizer, plant extract such as Chinese medicine, cationic, anionic, and non-ionic water-soluble polymer and polymer, adhesive/gel thickener, lower alcohol solvent, vitamin, amino acid, metal ion, blocking agent, preservative, pH adjusting agent, immersion agent, ultraviolet absorbent, anti-oxidizer, coloring agent, or spice.

The hair dye developed by the present invention uses highly safe natural colors (used in food and cosmetics), synthetic colors (used in food and cosmetics) and cosmetic colors to substitute the conventional genotoxic, carcinogenic, allergic chemical hair dye, such that users no longer need to worry about the issues of toxicity and allergicity when using the hair dye, so that this type of hair dyes can be used for both hair and skin or even beards, eyebrows, lips, and nails. The hair dyes manufactured by natural colors (used in food and cosmetics), synthetic colors (used in food and cosmetics) and cosmetic colors and used in various hair dyeing methods of the present invention can meet the standard of the present market, including the wash-resistance, sustainability, convenience and quickness, and some of the hair dyes disclosed in the present invention even exceed the present using standards. The natural colors (used in food and cosmetics), synthetic colors (used in food and cosmetics) and cosmetic colors of the present invention can be selected easily according to the color type, dye formula, and hair dyeing method to achieve the objectives of the permanent, semi-permanent, and temporary hair dyes. The hair dye can be manufactured in form of a hair dye cream, hair dye lotion, frost hair dye, hair dye gel, hair dye ointment, hair dye shampoo, foam hair dye, or spray hair dye. For special products, the natural colors (used in food and cosmetics), synthetic colors (used in food and cosmetics) and cosmetic colors of the present invention can be manufactured to produce a dyeing and perming hair dye and a hair dye that can change color with pH value. The colors of hair dyes developed by the present invention can be prepared in the same way of mixing drawing paints. For example, five colors such as red, yellow, blue, black and white colors are mixed, or even some more other colors are mixed to produce various color hair dyes required in the market, and then a predetermined hair dyeing method is used for dyeing hair, and the present invention has advantages such as simplicity, convenience, and innovation over various conventional hair dyes available in the market, and these advantages allow hair dyes to move towards a new milestone.

The present invention is illustrated by the following preferred embodiments, and it is noteworthy to point out that these preferred embodiments are provided for the purpose of illustrating the invention only, but not intended for limiting the scope of the present invention, and the invention is not limited to these preferred embodiments.

### 1. Preferred Embodiment of Oxidation Permanent Hair Dye Product

### (1) Composition of Hair Dye

Composition of first dose: food color ointment; composition of second dose: 6% of peroxide cream; and composition of third dose: monoethanolamine and potassium persulfate.

### (2) Dye Ointment:

Five kinds: Yellow 102 (FD&C Yellow No. 5), Orange 103 (FD&C Yellow No. 6), Red 104 (Acid Red 18), Magenta 105 (FD&C Red No. 3), and Blue 106 (FD&C Blue No. 1).

### (3) Hair Dyeing Method and Procedure

Hair dyeing method and procedure (I): The second dose and the third dose are mixed according to a predetermined proportion and prepared for use. A hair dyeing brush is used to coat the mixed dye onto each hair uniformly, and the mixed dye remains on the hair for 20 minutes, and then the dye on the hair is washed away by warm water, and a hair dyeing brush is used to apply the first dose onto each hair uniformly, and the first dose remains on the hair for 20 minutes, and then the hair is shampooed and rinsed to complete the hair dyeing process.

The hair dyeing method and procedure (II): The first dose, second dose and third dose are mixed according to a predetermined proportion and prepare for use. The mixed hair dye is coated uniformly onto each hair by a hair dyeing brush and stays on the hair for 20-30 minutes, and then the dye on the hair is washed by warm water, and the hair is shampooed and rinsed to complete the hair dyeing process.

Hair dyeing method and procedure (III): The second dose and the third dose are mixed according to a predetermined proportion and prepared for use. A hair dyeing brush is used to coat the mixed dye onto each hair uniformly, and the mixed dye remains on the hair for 10 minutes, and a hair dyeing brush is used to apply the first dose onto each hair uniformly and directly without washing away the mixed dye, and the first dose remains on the hair for 20 minutes, and then the hair is shampooed and rinsed to complete the hair dyeing process.

### (4) Testing Hair: White Hair and Black Hair

### (5) Ointment Formula of Hair dye: as Shown in Table 11.

The aforementioned five target color ointments are used for dyeing hair according to the hair dyeing method and procedures of I, II and III, and the testing hair is divided into white hair and black hair. After the hair is dyed, a commercial shampoo is used for washing and rinsing the aforementioned hair bundle repeatedly for 30 times, and a hair dryer is used for blowing dry the hair, and the color, of the dyed hair, dyeability and color retentability are evaluated by a visual inspection method according to the following standards, and the evaluation methods are listed in Table 12 and the final evaluation results are listed in Table 13.

TABLE 11
Ointment formula of oxidation type permanent hair dye

| | first dose | second dose | third dose |
|---|---|---|---|
| Composition/dosage form | (%) | (%) | (%) |
| food colors | 5 | | |
| Polyoxyethylene 1auryl ether | 6 | | |
| Polyoxyethylene(30)lanolin | 2 | | |
| Hexadecanol | 1.5 | | |
| Propanediol | 5 | | |
| Hexadecyl trimethylammonium | 1.5 | | |
| chloride | | | |
| Oleic acid | 10 | | |
| Water | Remaining | | |
| | quantity | | |
| hydrogen peroxide (30%) | | 20 | |
| Hexadecanol | | 1.5 | |
| Polyoxyethylene 1auryl ether | | 3 | |
| phosphoric acid | | 0.1 | |
| Sodium stannate | | 0.02 | |
| Water | | Remaining | |
| | | quantity | |
| Monoethanolamine (g) | | | 2 |
| Potassium persulfate (g) | | | 10 |

TABLE 12
Rating Method and Description of Hair Dye

| Evaluation | | |
|---|---|---|
| Method | | Grating and Description |
| Dyeability | A | Good and even dyeing results |
| | B | Not dyed properly or having uneven dyeing result. |
| | C | Not dyed yet or having uneven dyeing result |
| Color | A | Compared with the hair before shampoo, the hair |
| Retentability | | texture has no substantial difference. |
| | B | Compared with the hair before shampoo, some hairs |
| | | are discolored. |
| | C | Compared with the hair before shampoo, lots of hairs |
| | | are discolored. |

TABLE 13
Test results of oxidation type permanent hair dye

| | | | | | | | Preferred |
|---|---|---|---|---|---|---|---|
| Colors No./Hair | | | | | | | Embodiment |
| Dyeing Method | | 102 | 103 | 104 | 105 | 106 | No. |
| Method | Color after | Yellowish | Yellowish | Red | Burgundy | Bluish | First preferred |
| (I) | dyeing black | Brown | Orange | | | Green | embodiment |
| | hair | | | | | | |
| | Dyeability | A | A | A | A | A | |
| | Color | A | A | A | A | A | |
| | retentability | | | | | | |
| | Color after | Yellow | Orange | Red | Magenta | Blue | |
| | dyeing white | | | | | | |
| | hair | | | | | | |
| | Dyeability | A | A | A | A | A | |
| | Color | A | A | A | A | A | |
| | retentability | | | | | | |
| Method | Color after | Yellowish | Yellowish | Red | Burgundy | Bluish | Second |
| (II) | dyeing black | Brown | Orange | | | Green | preferred |
| | hair | | | | | | embodiment |
| | dyeability | A | A | A | A | A | |
| | Color | A | A | A | A | A | |
| | retentability | | | | | | |
| | Color after | Yellow | Orange | Red | Magenta | Blue | |
| | dyeing white | | | | | | |
| | hair | | | | | | |
| | dyeability | A | A | A | A | A | |
| | color | A | A | A | A | A | |
| | retentability | | | | | | |
| Method | Color after | Yellowish | Yellowish | Red | Burgundy | Bluish | Third preferred |
| (II) | dyeing black | Brown | Orange | | | Green | embodiment |
| | hair | | | | | | |
| | dyeability | A | A | A | A | A | |
| | color | A | A | A | A | A | |
| | retentability | | | | | | |
| | Color after | Yellow | Orange | Red | Magenta | Blue | |
| | dyeing white | | | | | | |
| | hair | | | | | | |
| | Dyeability | A | A | A | A | A | |
| | Color | A | A | A | A | A | |
| | retentability | | | | | | |

From the results listed in Table 13, the results obtained by using the hair dyeing methods I, II and III are the same, and the hair dyes are categorized as permanent hair dyes. The color obtained by dyeing white hair is the same as the color obtained by using food dyes. The color obtained by dyeing black hair, a mixed color of yellow and the dye color is obtained, since the 6% of peroxide cream can only discolor the black hair to a discoloration level up to a yellow or brown color, and the final color of the dyed hair will be a mixed color which is a normal result commonly known in this industry.

### 2. Preferred Embodiment of Disulfide Bond Reduction Type Permanent Hair Dye product

(1) Composition of hair dye: Food color ointment, disulfide bond reducer, and alkali.
(2) Dye: Five dyes including Yellow102 (FD&C Yellow No. 5), Orange 103 (FD&C Yellow No. 6), Red 104 (Acid Red 18), Magenta 105 (FD&C Red No. 3), and Blue 106 (FD&C Blue No. 1).
(3) Hair dyeing method and Procedure: A hair dyeing brush is used to coat the ointment of the hair dye on each hair uniformly, and the ointment stays on the hair for 20 minutes. For black or dark hair to be dyed, 6% of peroxide cream plus bleach is used to discolor the black color of the hair to yellow or brown (if the bleach stays on the hair for 30 minutes), and the bleach on the hair must be washed away by fresh water, and then a hair dyeing brush is used to coat the ointment of the hair dye onto each hair uniformly, and the ointment stays on the hair for 20 minutes, and a hair dryer or a steamer can be used for heating the hair during the stay period, and then this ointment is washed away by warm water, and then the hair is shampooed and rinsed to complete the hair dyeing process.
(4) Testing hair: white hair
(5) Ointment formula of hair dye: as shown in Table 14.

The aforementioned five target color ointments are used for dyeing hair according to the hair dyeing method and procedures of I, II and III, and white hair is used as the testing hair. After the hair is dyed, a commercial shampoo is used for washing and rinsing the aforementioned hair bundle repeatedly for 30 times, and a hair dryer is used for blowing dry the hair, and the color, of the dyed hair, dyeability and color retentability are evaluated by a visual inspection method according to the following standards, and the evaluation methods are listed in Table 12 and the final evaluation results are listed in Table 15.

TABLE 14
Formula of disulfide bond type permanent hair dyes

| Composition | Percentage by weight (%) |
|---|---|
| Food colors | 5 |
| Cysteine | 5 |
| Polyoxyethylene 1auryl ether | 6 |
| Polyoxyethylene (30)lanolin | 2 |
| Hexadecanol | 1.5 |
| Propanediol | 5 |
| Hexadecyl trimethylammonium chloride | 1.5 |
| Monoethanolamine | 4 |
| Water | Remaining Quantity |

TABLE 15
Test Result of disulfide bond type permanent hair dye

| Color No./ | | | | | | Preferred |
|---|---|---|---|---|---|---|
| hair dyeing | | | | | | embodiment |
| method | 102 | 103 | 104 | 105 | 106 | No. |
| Color after | Yellow | Orange | Red | Magenta | Blue | Preferred |
| dyeing | | | | | | embodiment |
| white hair | | | | | | 4 |
| Dyeability | A | A | A | A | A | |
| Color | A | A | A | A | A | |
| retentability | | | | | | |

From Table 15, the result shows that the dye used is categorized as the permanent hair dye, and the color obtained after dyeing white hair is the same as the color obtained by using food color dyes.

### 3. Preferred Embodiment of Metal Mordant Dyeing Type Permanent Hair Dye Product

(1) Composition of hair dye

Composition of first dose: this dose is a disulfide bond reducer for testing the white hair that is dyed, and 6% of a peroxide cream plus a bleach for testing the black hair that is dyed; composition of second dose: a metal ion ointment, which is ferric sulfate, magnesium chloride, or ferric chloride in this preferred embodiment; and composition of third dose: a food color ointment.
(2) Dye: Yellow 102 (FD&C Yellow No. 5), Orange 103 (FD&C Yellow No. 6), Red 104 (Acid Red 18), Magenta 105 (FD&C Red No. 3), and Blue 106 (FD&C Blue No. 1).
(3) Hair dyeing method and procedure

1. A hair dyeing brush is used to apply a first dose (disulfide bond reducer ointment) onto each hair uniformly, and the first dose stays on the hair for 10 minutes, and a hair dryer or a steamer can be used to heat the hair, and then this ointment on the hair is washed away by warm water, or the next step is performed directly without washing away the ointment on the hair. If the dyeing hair is black hair or dark hair, then this dose is replaced by 6% of peroxide cream plus a bleach, and the bleach stays on the hair for 30 minutes, and the bleach must be washed away by fresh water.

2. A hair dyeing brush is used for applying the second dose (metal ion ointment) onto each hair uniformly, and the second dose stays on the hair for 15 minutes, and a hair dryer or a steamer can be used for heating the hair during the stay period, and then this ointment on the hair is washed away by warm water.

3. A hair dyeing brush is used to apply the third dose (color dye ointment) on each hair uniformly, and the third dose stays on the hair for 15 minutes, and a hair dryer or a steamer can be used for heating the hair during the stay period, and then this ointment on the hair is washed away by warm water, and then the hair is shampooed and rinsed to complete the hair dyeing process.
(4) Testing hair: white hair and black hair.
(5) Ointment formula of hair dye: as shown in Table 16.

The aforementioned five target color ointments are used for dyeing hair according to the hair dyeing method and procedures of I, II and III, and white hair and black hair are used as the testing hair. After the hair is dyed, a commercial shampoo is used for washing and rinsing the aforementioned hair bundle repeatedly for 30 times, and a hair dryer is used for blowing dry the hair, and the color, of the dyed hair, dyeability and color retentability are evaluated by a visual inspection method according to the following standards, and the evaluation methods are listed in Table 12 and the final evaluation results are listed in Table 17.

TABLE 16
metal mordant dyeing permanent hair dye formula

| | first dose | second dose | third dose |
|---|---|---|---|
| Composition/dosage form | (%) | (%) | (%) |
| Metal ion | 3 | | |
| Sodium carboxymethyl | 1.2 | | |
| cellulose | | | |
| Sodium dodecyl sulfate | 2 | | |
| Propanediol | 10 | | |
| Triethanolamine | 0.2 | | |
| Water | Remaining | | |
| | Quantity | | |
| Cysteine | | 5 | |
| Polyoxyethylene 1auryl ether | | 6 | |
| Polyoxyethylene(30)lanolin | | 2 | |
| Hexadecanol | | 1.5 | |
| Propanediol | | 5 | |
| Hexadecyl | | 1.5 | |
| trimethylammonium chloride | | | |
| Monoethanolamine | | 4 | |
| Water | | Remaining | |
| | | Quantity | |
| Food colors | | | 5 |
| Polyoxyethylene 1auryl ether | | | 6 |
| Polyoxyethylene(30)lanolin | | | 2 |
| Hexadecanol | | | 1.5 |
| Propanediol | | | 5 |
| Hexadecyl | | | 1.5 |
| trimethylammonium chloride | | | |
| Oleic acid | | | 10 |
| Water | | | Remaining |
| | | | Quantity |

TABLE 17
Test results of metal mordant dyeing type permanent hair dye

| | | | | | | | preferred |
|---|---|---|---|---|---|---|---|
| Color No./hair | | | | | | | embodiment |
| dyeing method | | 102 | 103 | 104 | 105 | 106 | No. |
| Second- | Color after | Yellowish | Yellowish | Red | Burgundy | Bluish | preferred |
| dose metal | dyeing black | Brown | Orange | | | Green | embodiment 5 |
| ion: | hair | | | | | | |
| Aluminum | dyeability | A | A | A | A | A | |
| sulfate | color | A | A | A | A | A | |
| | retentability | | | | | | |
| | Color after | Yellow | Orange | Red | Magenta | Blue | |
| | dyeing white | | | | | | |
| | hair | | | | | | |
| | dyeability | A | A | A | A | A | |
| | color | A | A | A | A | A | |
| | retentability | | | | | | |
| Second-dose | Color after | Yellowish | Yellowish | Red | Burgundy | Bluish | preferred |
| metal ion: | dyeing black | Brown | Orange | | | Green | embodiment 6 |
| Magnesium | hair | | | | | | |
| chloride | dyeability | A | A | A | A | A | |
| | color | A | A | A | A | A | |
| | retentability | | | | | | |
| | Color after | Yellow | Orange | Red | Magenta | Blue | |
| | dyeing white | | | | | | |
| | hair | | | | | | |
| | dyeability | A | A | A | A | A | |
| | color | A | A | A | A | A | |
| | retentability | | | | | | |
| Second-dose | Color after | Yellowish | Yellowish | Red | Burgundy | Bluish | preferred |
| metal ion: | dyeing black | Brown | Orange | | | Green | embodiment 7 |
| Ferric | hair | | | | | | |
| chloride | dyeability | A | A | A | A | A | |
| | color | A | A | A | A | A | |
| | retentability | | | | | | |
| | Color after | Yellowish | Dark | Dark | Dark Red | Dark | |
| | dyeing white | Brown | Orange | Red | | Blue | |
| | hair | | | | | | |
| | dyeability | A | A | A | A | A | |
| | color | A | A | A | A | A | |
| | retentability | | | | | | |

From the result in Table 17, the test results of the hair dyeing test obtained by using three different kinds of metal ions show that the dyes are permanent hair dyes, and the color obtained by dyeing white hair is the same as the color obtained by using food colors as a dye, and the color of dyeing black hair becomes a mixed color of yellow and the dye color. The aforementioned test results are obtained when aluminum sulfate and magnesium chloride are used, and these results are the same as the oxidation test, since the 6% of peroxide cream can only discolor the black hair up to a discoloration level of yellow or brown, so that the final color of the dyed hair becomes a mixed color. However, if ferric chloride is used, the color of dyed hair obtained in the hair dyeing test is darker than the previous two, since ferric chloride has a yellowish brown color, and thus the color of the dyed hair will be darkened, and this result is classified as a normal result.

### 4. Preferred Embodiment of Ionic Bond Type Semi-Permanent Hair Dye Product

(1) Composition of hair dye: anion food colors, hair expander, penetration enhancer, and acid.
(2) Dye: Yellow 102 (FD&C Yellow No. 5), Orange 103 (FD&C Yellow No. 6), Red 104 (Acid Red 18), Magenta 105 (FD&C Red No. 3), and Blue 106 (FD&C Blue No. 1).
(3) Hair dyeing method and procedure: A hair dyeing brush is used to coat the ointment of the hair dye onto each hair, and the ointment stays on the hair for 20 minutes. For black hair or dark hair to be dyed, 6% of peroxide cream plus a bleach is used to discolor the black hair to yellow or brown (and the bleach stays on the hair for 30 minutes), and it is necessary to wash away the bleach by fresh water, and then a hair dyeing brush is used to coat the ointment of the hair dye onto each hair, and the ointment stays on the hair for 20 minutes, and a hair dryer or a steamer can be used to heat the hair during the stay period, and then this ointment is washed away by warm water, and then the hair is shampooed and rinsed to complete the hair dyeing process.
(4) Testing hair: white hair
(5) Ointment formula of hair dye: as shown in Table 18.

The aforementioned five target color ointments are used for dyeing hair according to the hair dyeing method and procedure, and white hair is used for the hair dyeing test , After the hair is dyed, a commercial shampoo is used for washing and rinsing the aforementioned hair bundle repeatedly for 15 times, and a hair dryer is used for blowing dry the hair, and the color, of the dyed hair, dyeability and color retentability are evaluated by a visual inspection method according to the following standards, and the evaluation methods are listed in Table 12 and the final evaluation results are listed in Table 19.

TABLE 18
Ionic bond method semi-permanent hair dye formula

| Composition | Percentage by weight (%) |
|---|---|
| Food colors | 5 |
| Isopropanol | 5 |
| Benzyl alcohol | 5 |
| Polyoxyethylene lauryl ether | 6 |
| Polyoxyethylene(30)lanolin | 2 |
| Propanediol | 5 |
| Hexadecyl trimethylammonium chloride | 1.5 |
| Citric acid | 1 |
| Water | Remaining Quantity |

TABLE 19
Test result of ionic bond type semi-permanent hair dye

| Color No./ | | | | | | Preferred |
|---|---|---|---|---|---|---|
| hair dyeing | | | | | | embodiment |
| method | 102 | 103 | 104 | 105 | 106 | No. |
| Color after | Yellow | Orange | Red | Magenta | Blue | Preferred |
| dyeing | | | | | | embodiment |
| white hair | | | | | | 8 |
| Dyeability | A | A | A | A | A | |
| Color | A | A | A | A | A | |
| retentability | | | | | | |

From the results listed in Table 19, the result of the dyed hair shows that the dye is categorized as the semi-permanent hair dye, and the dyed color of the white hair is the same as the color obtained by using food color dyes.

### 5. Preferred Embodiment of Hair Dye Prepared by Five Primitive Color Ointments

(1) Composition of hair dye: The hair dye is prepared with a composition according to the oxidation, disulfide bond reduction, and metal mordant dyeing.
(2) Dye: Five basic colors such as Yellow 102 (FD&C Yellow No. 5), Orange 103 (FD&C Yellow No. 6), Red 104 (Acid Red 18), Magenta 105 (FD&C Red No. 3) and Blue 106 (FD&C Blue No. 1) are mixed according to a predetermined proportion as shown in Table 20.
(3) Hair dyeing method and procedure: The hair is dyed according to the oxidation, disulfide bond reduction, or metal mordant dyeing type hair dyeing procedure.
(4) Testing hair: white hair and black hair.

The dye ointment prepared according to Table 20 is used for dyeing hair according to the oxidation, disulfide bond reduction, or metal mordant dyeing procedure, and white hair and black hair are used as the testing hair. After the hair is dyed, a commercial shampoo is used for washing and rinsing the aforementioned hair bundle repeatedly for 30 times, and a hair dryer is used for blowing dry the hair, and the color, of the dyed hair, dyeability and color retentability are evaluated by a visual inspection method according to the following standards, and the evaluation methods are listed in Table 12 and the final evaluation results are listed in Table 21.

TABLE 20
Table of preparing colors

| | | | | | | Color after | Pantone |
|---|---|---|---|---|---|---|---|
| Dye No. | 102 | 103 | 104 | 105 | 106 | mixing | No. |
| 51 | 2 | 1 | 0 | 0 | 2 | Fresh Green | 348C |
| 66 | 0 | 0 | 0 | 1 | 4 | Dark Purple | 526C |
| 96 | 2 | 0 | 2 | 0 | 1 | Brown | 498C |
| 12 | 1 | 1 | 2 | 1 | 0 | Orange | 1787C |
| 85 | 0 | 0 | 2 | 2 | 1 | Purplish Red | 227C |

TABLE 21
Test results of hair dye prepared by mixing five primitive color ointments

| | | | | | | | Preferred |
|---|---|---|---|---|---|---|---|
| | | | | | | | embodiment |
| Dye No. | | No. 51 | No. 66 | No. 96 | No. 12 | No. 85 | No. |
| oxidation | Color after | Fresh | Dark | Brown | Orange | Purplish | Preferred |
| | dyeing black | Green | Purple | | | Red | embodiment 9 |
| | hair | | | | | | |
| | dyeability | A | A | A | A | A | |
| | color | A | A | A | A | A | |
| | retentability | | | | | | |
| | Color after | Fresh | Dark | Brown | Orange | Purplish | |
| | dyeing white | Green | Purple | | | Red | |
| | hair | | | | | | |
| | dyeability | A | A | A | A | A | |
| | color | A | A | A | A | A | |
| | retentability | | | | | | |
| disulfide | Color after | Fresh | Dark | Brown | Orange | Purplish | Preferred |
| bond | dyeing black | Green | Purple | | | Red | embodiment |
| reduction | hair | | | | | | 10 |
| | dyeability | A | A | A | A | A | |
| | color | A | A | A | A | A | |
| | retentability | | | | | | |
| | Color after | Fresh | Dark | Brown | Orange | Purplish | |
| | dyeing white | Green | Purple | | | Red | |
| | hair | | | | | | |
| | dyeability | A | A | A | A | A | |
| | color | A | A | A | A | A | |
| | retentability | | | | | | |
| metal | Color after | Fresh | Dark | Brown | Orange | Purplish | preferred |
| mordant | dyeing black | Green | Purple | | | Red | embodiment |
| dyeing | hair | | | | | | 11 |
| | dyeability | A | A | A | A | A | |
| | color | A | A | A | A | A | |
| | retentability | | | | | | |
| | Color after | Fresh | Dark | Brown | Orange | Purplish | |
| | dyeing white | Green | Purple | | | Red | |
| | hair | | | | | | |
| | dyeability | A | A | A | A | A | |
| | color | A | A | A | A | A | |
| | retentability | | | | | | |

From the results listed in Table 21, the dye prepared by mixing colors can be dyed on white hair and black hair successfully regardless of the hair dyeing method used, and the result of the dyed color is the same as those obtained by various different hair dyeing methods without any difference, and the dye is categorized as the permanent hair dye.

### 6. Preferred Embodiment of Dyeing and Perming Products

(1) Composition of two-dose dyeing and perming product

First dose: food color ointment, disulfide bond reducer, and alkali; second dose: disulfide bond oxidizer (6% of peroxide cream).
(2) Dye: Magenta 105 (FD&C Red No. 3); or black dye.

The black dye is prepared by the following method: The proportion of Yellow 102 (FD&C Yellow No. 5): Orange 103 (FD&C Yellow No. 6): Red 104 (Acid Red 18): Blue 106 (FD&C Blue No. 1) is equal to 4:2:4:2.
(3) Dyeing and perming method and procedure: Three methods such as thermoplastic method, lotion wrap method, and cold perming method can be used for the testing, and the operating procedure is shown in Table 22.
(4) Testing hair: white hair and black hair.
(5) Ointment formula: as shown in Tables 23 and 23-1.
(6) Testing effect: as listed in Tables 23-2 and 23-3.

TABLE 22
Operating procedure of thermoplastic method,
lotion wrap method and cold perming method

| Perm method/ | Thermoplastic | Lotion wrap | Cold perming |
|---|---|---|---|
| procedure | method | method | method |
| Step 1 | Shampoo | Shampoo | Shampoo |
| Step 2 | Apply first dose | Apply first dose | Apply first |
| | | | dose |
| Step 3 | Rinse | Set rollers | Rinse |
| Step 4 | Set rollers | Heat, shape and dry | Set rollers |
| Step 5 | Heat, shape and | Apply second dose | Apply second |
| | dry | | dose |
| Step 6 | Apply second | Remove rollers | Remove rollers |
| | dose | | |
| Step 7 | Remove rollers | Rinse | Rinse |
| Step 8 | Rinse | | |

TABLE 23
Formula of two-dose dyeing and perming products (preferred embodiments 12, 13
and 14)

| | Thermoplastic | | Lotion wrap | | Cold perming | |
|---|---|---|---|---|---|---|
| | method | | method | | method | |
| | (preferred | | (preferred | | (preferred | |
| | embodiment 12) | | embodiment 13) | | embodiment 14) | |
| Method/ | | second | | second | | second |
| Composition | first dose | dose | first dose | dose | first dose | dose |
| 105 food colors | 5 | | 5 | | 5 | |
| cysteine | 5 | | 5 | | 5 | |
| Polyoxyethylene lauryl | 6 | | 6 | | 6 | |
| ether | | | | | | |
| Polyoxyethylene(30)lanolin | 2 | | 2 | | 2 | |
| Hexadecanol | 1.5 | | 1.5 | | 1.5 | |
| Propanediol | 5 | | 5 | | 5 | |
| Hexadecyl | 1.5 | | 1.5 | | 1.5 | |
| trimethylammonium | | | | | | |
| chloride | | | | | | |
| monoethanolamine | 4 | | 4 | | 4 | |
| Ethanethiol acid | | | 5 | | 6 | |
| sodium sulfite | | | | | 2 | |
| Water | Remaining | | Remaining | | Remaining | |
| | quantity | | quantity | | quantity | |
| hydrogen peroxide (30%) | | 20 | | 20 | | 20 |
| Hexadecanol | | 1.5 | | 1.5 | | 1.5 |
| Polyoxyethylene lauryl | | 3 | | 3 | | 3 |
| ether | | | | | | |
| phosphoric acid | | 0.1 | | 0.1 | | 0.1 |
| Sodium stannate | | 0.02 | | 0.02 | | 0.02 |
| Water | | Remaining | | Remaining | | Remaining |
| | | quantity | | quantity | | quantity |

TABLE 23-1
two-dose dyeing and perming product formula (preferred embodiment 12-1, 13-1,
14-1)

| | thermoplastic | | lotion wrap | | cold | |
|---|---|---|---|---|---|---|
| | method | | method | | perming method | |
| | (preferred | | (preferred | | (preferred | |
| | embodiment 12-1) | | embodiment 13-1) | | embodiment 14-1) | |
| Method/ | | second | | second | | second |
| Composition | first dose | dose | first dose | dose | first dose | dose |
| Yellow 102 | 1 | | 1 | | 1 | |
| Orange 103 | 0.5 | | 0.5 | | 0.5 | |
| Red 104 | 1 | | 1 | | 1 | |
| Blue106 | 0.5 | | 0.5 | | 0.5 | |
| cysteine | 5 | | 5 | | 5 | |
| Polyoxyethylene lauryl | 6 | | 6 | | 6 | |
| ether | | | | | | |
| Polyoxyethylene(30)lanolin | 2 | | 2 | | 2 | |
| Hexadecanol | 1.5 | | 1.5 | | 1.5 | |
| Propanediol | 5 | | 5 | | 5 | |
| Hexadecyl | 1.5 | | 1.5 | | 1.5 | |
| trimethylammonium | | | | | | |
| chloride | | | | | | |
| monoethanolamine | 4 | | 4 | | 4 | |
| Ethanethiol acid | | | 5 | | 6 | |
| sodium sulfite | | | | | 2 | |
| Water | Remaining | | Remaining | | Remaining | |
| | quantity | | quantity | | quantity | |
| hydrogen peroxide (30%) | | 20 | | 20 | | 20 |
| Hexadecanol | | 1.5 | | 1.5 | | 1.5 |
| Polyoxyethylene lauryl | | 3 | | 3 | | 3 |
| ether | | | | | | |
| phosphoric acid | | 0.1 | | 0.1 | | 0.1 |
| Sodium stannate | | 0.02 | | 0.02 | | 0.02 |
| Water | | Remaining | | Remaining | | Remaining |
| | | quantity | | quantity | | quantity |

TABLE 23-2
Dye using Magenta 105

| | Perming Nature | | | | Dyeing Nature | | |
|---|---|---|---|---|---|---|---|
| | | | cold | | | | cold |
| Item | thermoplastic | lotion wrap | perming | Item | thermoplastic | lotion wrap | perming |
| Class | method | method | method | Class | method | method | method |
| Elasticity | Good | Good | Good | Color after | Burgundy | Burgundy | Burgundy |
| | | | | dyeing | | | |
| | | | | black hair | | | |
| Hair | Good | Good | Good | Dyeability | A | A | A |
| texture | | | | | | | |
| Odor of | None | None | None | Color | A | A | A |
| Remained | | | | retentability | | | |
| Chemicals | | | | | | | |
| Texture | Good | Good | Good | Color after | Pink | Pink | Pink |
| after perm | | | | dyeing | | | |
| | | | | white hair | | | |
| Luster | Good | Good | Good | Dyeability | A | A | A |
| after perm | | | | | | | |
| Curl | >95% | >95% | >90% | Color | A | A | A |
| achievement | | | | retentability | | | |
| rate | | | | | | | |

TABLE 23-3
Using black dye

| | Perming Nature | | | | Dyeing Nature | | |
|---|---|---|---|---|---|---|---|
| | | | cold | | | | cold |
| Item | thermoplastic | lotion wrap | perming | Item | thermoplastic | lotion wrap | perming |
| Class | method | method | method | Class | method | method | method |
| Elasticity | Good | Good | Good | Color after | Black | Black | Black |
| | | | | dyeing | | | |
| | | | | black hair | | | |
| Hair | Good | Good | Good | Dyeability | A | A | A |
| texture | | | | | | | |
| Odor of | None | None | None | Color | A | A | A |
| Remained | | | | retentability | | | |
| Chemicals | | | | | | | |
| Texture | Good | Good | Good | Color after | Black | Black | Black |
| after perm | | | | dyeing | | | |
| | | | | white hair | | | |
| Luster | Good | Good | Good | Dyeability | A | A | A |
| after perm | | | | | | | |
| Curl | >95% | >95% | >90% | Color | A | A | A |
| achievement | | | | retentability | | | |
| rate | | | | | | | |

### 7. Preferred Embodiment of Foam Hair Dye

(1) Composition of Foam Hair Dye
(I) One-dose form: food color ointment, foaming agent, and alkali. (II) Two-dose form: First dose: food color ointment, foaming agent, and alkali; second dose: 6% of peroxide cream.

(2) Operating Procedure
(I) One-dose operating procedure: The dye ointment is poured into a bottle and shaken for 30 seconds, and gloves are put on to coat the foam onto the hair, and allow the foam to stay on the hair for 30 minutes. For dyeing black hair or dark hair, a bleach is added to 6% of the peroxide cream, and the color of the black hair is discolored to yellow or brown (let the bleach stay for 30 minutes), and then the bleach is washed away by fresh water. The gloves are put on coat the foam onto the hair and allow the foam to stay on the hair for 30 minutes, and then this ointment of the hair is washed away by warm water, and then the hair is shampooed and rinsed to complete the hair dyeing process.
(II) The two-dose operating procedure: The dye ointment in the bottle 1 is poured into the bottle 2 and shaken for 30 seconds, and gloves are put on to coat the foam on the hair and allow the foam to stay on the hair for 30 minutes, and this ointment on the hair is washed away by warm water, and then the hair is shampooed and rinsed to complete the hair dyeing process.
   (3) Ointment Formula: as shown in Table 24.

TABLE 24
Formula of foam hair dye (Preferred embodiment 15)

| | | Non-aerosol | |
|---|---|---|---|
| | Non-aerosol | foam hair dye | |
| | foam hair dye | (two-dose form) | |
| Dosage form/Composition | (one-dose form) | Bottle 1 | Bottle 2 |
| Food colors | 5 | 5 | |
| Benzyl alcohol | 5 | | |
| Hexadecyl | 1.5 | 1.5 | |
| trimethylammonium chloride | | | |
| PEG-7 Coconut acid glyceride | 0.5 | | |
| Polysorbate 20 | 2 | 10 | |
| Foaming agent | 2 | 5 | |
| Water | Remaining | Remaining | |
| | Quantity | Quantity | |
| Monoethanolamine | | 4 | |
| Cocamide DAE | | 3 | |
| Oleic alcohol | | 5 | |
| Hydrogen peroxide (30%) | | | 20 |
| Hexadecanol | | | 1.5 |
| Polyoxyethylene lauryl ether | | | 3 |
| Phosphoric acid | | | 0.1 |
| Sodium stannate | | | 0.02 |
| Water | | | Remaining |
| | | | Quantity |

### 8. Preferred Embodiment of Hair Dye Shampoo

(1) Composition of hair dye shampoo
   First dose: food colors, anion surfactant, anti-oxidizer, and alkali.
   Second dose: 6% of peroxide cream.
(2) Operating procedure: The hair is moistened by water first, and then the first dose and the second dose are poured on hands and mixed, and the aforementioned ointments are applied onto the hair by a shampooing method, and then the hair is shampooed by warm water to wash away the shampoo on the hair to complete the hair dyeing process.
(3) Ointment formula: as shown in Table 25.

TABLE 25
Hair dye shampoo formula (preferred embodiment 16)

| Composition | first dose (%) | second dose (%) |
|---|---|---|
| Food colors | 5 | |
| Fatty acid methylester sodium | 5 | |
| aminoguanidine sulfate | | |
| Sodium alcohol ether sulphate | 12 | |
| Cocoamide DEA | 2 | |
| Isoascorbic acid | 0.5 | |
| Sodium sulfite | 0.5 | |
| Triethanolamine | 4 | |
| Water | Remaining | |
| | Quantity | |
| Hydrogen peroxide (30%) | | 20 |
| Hexadecanol | | 1.5 |
| Polyoxyethylene lauryl ether | | 3 |
| Hexadecyl trimethylammonium | | 1.5 |
| chloride | | |
| Phosphoric acid | | 0.1 |
| Sodium stannate | | 0.02 |
| Water | | Remaining |
| | | Quantity |

### 9. Preferred Embodiment of Hair Dye that Changes Colors with Different pH Values

(1) Composition of hair dye:

The composition is prepared according to the oxidation, disulfide bond reduction, and metal mordant dyeing.
(2) Colors used: Grape skin color (Color Change Range: Red to purple), carmine color (Color Change Range: Red to orange), anatto color (Color Change Range: yellow to orange).
(3) Hair dyeing method and procedure: The hair is dyed according to the oxidation, disulfide bond reduction, or metal mordant dyeing type hair dyeing procedure.
(4) Testing hair: white hair.
(5) pH values of buffer solutions: pH=2.0, pH=4.0, pH=7.0, and pH=9.0.

The grape skin colors, carmine colors, annatto colors are used for preparing the ointments of the hair dye, and the hair is dyed according to the oxidation, disulfide bond reduction, or metal mordant dyeing procedure, and white hair is used for the hair dyeing test. After the hair is dyed, a commercial shampoo is used for washing and rinsing the aforementioned hair bundle repeatedly for 30 times, and a hair dryer is used for blowing dry the hair, and buffer solutions of different pH values are sprayed onto the hair bundles of the dyed hair, and then the hair dryer is used for blowing dry the hair again, and the color of the dyed hair, dyeability and color retentability are evaluated by a visual inspection method according to the standards below, and the final evaluation results are listed in Table 26.

TABLE 26
Test Results of Hair Dye that Changes Colors with Different pH values

| | | Grape skin | Carmine | Annatto | Preferred |
|---|---|---|---|---|---|
| Color of the dye | | colors | colors | colors | embodiment No. |
| Oxidation | Color of recently | Purplish Blue | Red | Yellow | Preferred |
| | dyed hair | | | | embodiment |
| | pH = 2.0 | Red | Red | Yellow | 17 |
| | pH = 4.0 | Pink | Red | Yellow | |
| | pH = 7.0 | Purple | Orange | Yellowish | |
| | | | | Orange | |
| | pH = 9.0 | Bluish Purple | Orange | Orange | |
| Disulfide bond | Color of recently | Purplish Blue | Red | Yellow | Preferred |
| reduction | dyed hair | | | | embodiment |
| | pH = 2.0 | Red | Red | Yellow | 18 |
| | pH = 4.0 | Pink | Red | Yellow | |
| | pH = 7.0 | Purple | Orange | Yellowish | |
| | | | | Orange | |
| | pH = 9.0 | Bluish Purple | Orange | Orange | |
| Metal mordant | Color of recently | Purplish Blue | Red | Yellow | Preferred |
| dyeing | dyed hair | | | | embodiment |
| | pH = 2.0 | Red | Red | Yellow | 19 |
| | pH = 4.0 | Pink | Red | Yellow | |
| | pH = 7.0 | Purple | Orange | Yellowish | |
| | | | | Orange | |
| | pH = 9.0 | Bluish Purple | Orange | Orange | |

From the results listed in Table 26, the color change range of the hair color and the properties of color molecules are the same, and the experiments show that the feature of this color change can be repeated for many times without fading.

### 10. Referred Embodiment of Oxidation Type Permanent Hair Dye Product orimarily using Cosmetic Colors

(1) Composition of hair dye

Composition of first dose: cosmetic color ointment; composition of second dose: 6% of peroxide cream; and composition of third dose: monoethanolamine and potassium persulfate. (2) Dye ointment: D&C Yellow No. 10, Ext. D&C Orange No.3, FD&C Red No. 4, D&C Blue No. 6 are four cosmetic colors and mixed in a proportion of 1:1:1:2, and the color obtained after mixing these colors is bluish green with a Pantone color number of 5467C.
(3) Hair dyeing method and procedure

Hair dyeing method and procedure (I): The second dose and the third dose are mixed according to a predetermined proportion and prepared for use. A hair dyeing brush is used to coat the mixed dye onto each hair uniformly, and the mixed dye remains on the hair for 20 minutes, and then the dye on the hair is washed away by warm water, and then a hair dyeing brush is used to apply a first dose onto each hair uniformly, and the first dose remains on the hair for 20 minutes, and then the hair is shampooed and rinsed to complete the hair dyeing process.

Hair dyeing method and procedure (II): The first dose, the second dose and the third dose are mixed according to a predetermined proportion and prepared for use. A hair dyeing brush is used to coat the mixed dye onto each hair uniformly, and the mixed dye remains on the hair for 20-30 minutes, and then the dye on the hair is washed away by warm water, and then the hair is shampooed and rinsed to complete the hair dyeing process.

Hair dyeing method and procedure (III): The second dose and the third dose are mixed according to a predetermined proportion and prepared for use. A hair dyeing brush is used to coat the mixed dye onto each hair uniformly, and the mixed dye remains on the hair for 10 minutes, and a hair dyeing brush is used to apply the first dose onto each hair uniformly and directly without washing the hair, and the first dose remains on the hair for 20 minutes, and then the hair is shampooed and rinsed to complete the hair dyeing process.
(4) Testing hair: white hair and black hair
(5) Ointment formula of hair dye: as shown in Table 27.

The aforementioned five target color ointments are used for dyeing hair according to the hair dyeing method and procedures I, II and III, and the testing hair is divided into white hair and black hair. After the hair is dyed, a commercial shampoo is used for washing and rinsing the aforementioned hair bundle repeatedly for 30 times, and a hair dryer is used for blowing dry the hair, and the color of the dyed hair, dyeability and color retentability are evaluated by a visual inspection method according to the standards as shown in Table 28, and the final evaluation results are listed in Table 29.

TABLE 27
Formula of oxidation type permanent hair dye ointment

| | first dose | second dose | third dose |
|---|---|---|---|
| Composition/dosage form | (%) | (%) | (%) |
| D&C Yellow 10 | 0.5 | | |
| Ext. D&C Orange 3 | 0.5 | | |
| FD&C Red 4 | 0.5 | | |
| D&C Blue 6 | 2 | | |
| Polyoxyethylene lauryl ether | 6 | | |
| Polyoxyethylene(30)lanolin | 2 | | |
| Hexadecanol | 1.5 | | |
| Propanediol | 5 | | |
| Hexadecyl trimethylammonium | 1.5 | | |
| chloride | | | |
| Oleic acid | 10 | | |
| Water | Remaining | | |
| | Quantity | | |
| hydrogen peroxide (30%) | | 20 | |
| Hexadecanol | | 1.5 | |
| Polyoxyethylene lauryl ether | | 3 | |
| phosphoric acid | | 0.1 | |
| Sodium stannate | | 0.02 | |
| Water | | Remaining | |
| | | Quantity | |
| monoethanolamine(g) | | | 2 |
| potassium persulfate (g) | | | 10 |

TABLE 28
Evaluation method and description of hair dyes

| Evaluation | | |
|---|---|---|
| method | Evaluation Grade and Description | |
| Dyeability | A | Uniform and good dyeing color\ |
| | B | Incomplete or non-uniform dyeing |
| | C | Not dyed or dyed non-uniformly |
| Color | A | Compared hair bundles before shampoo, there is no |
| retentability | | substantial difference. |
| | B | Compared hair bundles before shampoo, minor fading |
| | | can be observed. |
| | C | Compared hair bundles before shampoo, major fading |
| | | can be observed. |

TABLE 29
Test result of oxidation type permanent hair dye

| | | Experiment | Preferred |
|---|---|---|---|
| Color No./Hair dyeing method | | Result | embodiment No. |
| Method | Color after dyeing black hair | Bluish Green | Preferred |
| (I) | dyeability | A | embodiment 20 |
| | Color retentability | A | |
| | Color after dyeing white hair | Blue | |
| | dyeability | A | |
| | Color retentability | A | |
| Method | Color after dyeing black hair | Bluish Green | Preferred |
| (II) | Dyeability | A | embodiment 21 |
| | Color retentability | A | |
| | Color after dyeing white hair | Blue | |
| | Dyeability | A | |
| | Color retentability | A | |
| Method | Color after dyeing black hair | Bluish Green | Preferred |
| (II) | Dyeability | A | embodiment 22 |
| | Color retentability | A | |
| | Color after dyeing white hair | Blue | |
| | Dyeability | A | |
| | Color retentability | A | |

From the results listed in Table 29, the results obtained by using the hair dyeing methods I, II and III are the same, and the hair dyes are categorized as permanent hair dyes, and the color obtained by dyeing white hair approaches a bluish green color, and the color obtained by dyeing black hair approaches a darker bluish green color.

### REFERENCE

1. AMES, B N., KAMMEN, H O., YAMASAKI, E. Hair dyes are mutagenic: Identification of a variety of mutagenic ingredients. Proc Nat Acad Sci USA. 1975, 72(6), 2423-2427.
2. HANLON, J. Tint of suspicion. New Scientist. 1978, 78, 352-357.
3. CHUNG, K. T., MURDOCU, C. A., ZHOU, Y. G. Effects of the nitro-group on the mutagenicity and toxicity of some benzamines. Environ and Mol Mutagen. 1996, 27(1), 67-74.
4. CHUNG, K. T., HUGHES, T. J., CLAXTON, L. D. Comparison of the mutagenic specificity induced by four nitro-group-containing aromatic amines in Salmonella typhimurium his genes. Mutat Res. 2000, 465 (2), 165-171.
5. CHENS, S. U., CHING, T. Y., CHUNG, K. T. Base-pair mutation caused by four nitro-group-containing aromatic amines in Salmonella typhimurium TA100, TA104, TA4001 and TA4006. Mutat Res. 1997, 3 95(3), 223-227.
6. SHAHIN, M. M. Structure-activity relationships within various series of p-phenylenediamine derivatives. Mutat Res. 1994, 307(1), 83-93.
7. DAI, Xiudao, YANG Wei, ZHOU De Hao. Study on the Effect of Oxidation Hair Dye on DNA in Large Rat Skin Cells. Chinese Journal of Public Health. 2000, 16(10), 905-906.
8. SHAHIN, M. M., ROUERS, D., BUGAUT, A. Structure-activity relationship with a series of 2,4-diaminoalkoxybenzene compounds. Mutat Res. 1980, 79(3), 289-306.
9. SHAHIN, M. M., CHOPY, C., LEGUESNE, N. Comparisons of mutation induction by six monocyclic aromatic amines in Salmonella typhimurium tester strains TA97. TA1537. TA1538. Environ Mutag en. 1985, 7(4), 535-546.
10. SHAHIN, M. M., BUGAUT, A., KALOPISSIS, G. Mutagenicity of aminonitrophenol compounds in Salmonella typhimurium: a study of structure-activity relationships. Int J Cosmetic Sci.1982, 4(1), 25-35.
11. SHAHIN, M. M., BUGAUT, A., KALOPISSIS, G. Structure-activity relationship with a series of m-diaminobenzene derivatives. Mutat Res. 1980, 78(1), 25-31.
12. SHAHIN, M. M., C HOPY, C. Studies on the mutagen: city of monocyclic aromatic amines in Salmonella typhimurium: detoxification effects of liver S9. Fourth Int Conf Environ Mutagens. 1985, 229.
13. WATANABE, T., HIRAYAMA, T., FUKUI, S. Phenarzine derivatives as the mutagenic reaction product from o-or m-phenylenediamine derivatives with hydrogen peroxide. Mutat Res. 1989, 227(3), 135-145.
14. SHAHIN, M. M. Structure-activity relationships within various series of p-phenylenediamine derivatives. Mutat Res. 1994, 307(2), 83-93.

1. A hair dye, comprising:
(a) a dye, being one selected from the collection of a natural color (used in food and cosmetics), a synthetic color (used in food and cosmetics) and a cosmetic color; and
(b) a cuticle opener.

2. The hair dye according to claim 1, being a permanent hair dye, and comprising:
(a) a synthetic color of the hair dye (used in food and cosmetics);
(b) an inorganic peroxide or an organic peroxide, a disulfide bond reducer, or a bleaching agent used as the cuticle opener; and
(c) a freely selected metal ion and/or alkali.

3. The hair dye according to claim 2, wherein the synthetic color is one selected from the collection of FD&C Yellow No. 5, FD&C Yellow No. 6, Acid Red 18, FD&C Red No. 3, FD&C Blue No. 1 and any combination of the above, and the cosmetic colors is one selected from the collection of D&C Yellow No. 10, Ext. D&C Orange No.3, FD&C Red No. 4, D&C Blue No. 6 and any combination of the above.

4. The hair dye according to claim 2, wherein the inorganic peroxide is hydrogen peroxide or aqueous hydrogen peroxide.

5. The hair dye according to claim 2, wherein the disulfide bond reducer is one selected from the collection of D, L-cysteine or its salts, N-acetyl-D, L-cysteine or its salts, thiol acid or its salts or esters, thiolactic acid or its salts, sulfurous acid or its salts, ascorbic acid or its salts, L-ascorbic acid (vitamin C) or its salts, and any combination of the above.

6. The hair dye according to claim 2, wherein the metal ion is one selected from magnesium cation, aluminum cation, iron cation and zinc cation.

7. The hair dye according to claim 2, wherein the color (used in food and cosmetics) occupies 1% to 10% by weight of the hair dye, and the cuticle opener occupies 1% to 12% by weight of the hair dye and the metal ions occupy 1% to 10% by weight of the hair dye, and the alkali occupies 1% to 10% by weight of the hair dye.

8. The hair dye according to claim 1, being a semi-permanent hair dye and comprising:
(a) a natural or synthetic color (used in food and cosmetics) used as a hair dye;
(b) a hair penetration enhancer and/or a hair expander; and
(c) an acid or an alkali.

9. The hair dye according to claim 8, wherein the synthetic food color is one selected from the collection of FD&C Yellow No. 5, FD&C Yellow No. 6, Acid Red 18, FD&C Red No. 3, FD&C Blue No. 1 and any combination of the above, and the synthetic cosmetic color is one selected from the collection of D&C Yellow No. 10, Ext. D&C Orange No.3, FD&C Red No. 4, D&C Blue No. 6 and any combination of the above.

10. The hair dye according to claim 8, wherein the hair penetration enhancer and/or hair expander is one selected from the collection of benzyl alcohol, isopropanol, N-methyl-2-pyrrolidone, propylene carbonate, sodium sulfate anhydrate, benzoic acid, formic acid, urea, guanidine, sodium dodecyl sulfate, sodium N-dodecanoylsalcosinate, Triton X-100, and dodecylazacycloneptan-2-one 1.

11. The hair dye according to claim 8, wherein the color (used in food and cosmetics) occupies 1% to 10% by weight of the hair dye, and the hair penetration enhancer and/or hair expander occupies 1% to 15% by weight of the hair dye and the acid or alkali occupies 1% to 10% by weight of the hair dye.

12. The hair dye according to claim 1, being a temporary hair dye, and comprising a natural color or a synthetic color (used in food and cosmetics).

13. The hair dye according to claim 12, wherein the synthetic food color is one selected from the collection of FD&C Yellow No. 5, FD&C Yellow No. 6, Acid Red 18, FD&C Red No. 3, FD&C Blue No. 1, and any combination of the above, and the synthetic cosmetic color is one selected from the collection of D&C Yellow No. 10, Ext. D&C Orange No.3, FD&C Red No. 4, D&C Blue No. 6, and a combination of the above.

14. The hair dye according to claim 12, wherein the colors (used in food and cosmetics) occupies 1% to 10% by weight of the hair dye.

15. The hair dye according to any one of claims 1, being in a form selected from the collection of a hair dye cream, a hair dye lotion, a frost hair dye, a hair dye gel, a hair dye ointment, a hair dye shampoo, a foam type hair dye, a spray type hair dye, a perm combined with a hair dye, and a hair dye changing a color thereof with a pH value.

16. The hair dye according to claim 15, combined with a perm agent to produce a hair dyeing and perm agent, and comprising:
(a) a disulfide bond reducer;
(b) a natural or synthetic color (used in food and cosmetics) used as a dye; and
(c) a disulfide bond oxidizer.

17. The hair dye according to claim 16, wherein the synthetic food color is one selected from the collection of FD&C Yellow No. 5, FD&C Yellow No. 6, Acid Red 18, FD&C Red No. 3, FD&C Blue No. 1 and any combination of the above, and the synthetic cosmetic color is one selected from the collection of D&C Yellow No. 10, Ext. D&C Orange No.3, FD&C Red No. 4, D&C Blue No. 6 and any combination of the above.

18. The hair dye according to claim 15, wherein the hair dye changes its color with a change of pH value, and comprises:
(a) a natural color (used in food and cosmetics) used as a dye;
(b) an acidic or alkaline buffer having a pH value from 2.0 to 10.0; and
(c) a metal ion.

19. The hair dye according to claim 18, wherein the natural color (used in food and cosmetics) is one selected from the collection of a grape skin color, a carmine color, a rouge orange color, anthocyanin, carminic acid, betanin, vulgaxanthin, santalin, bixin, norbixin, and a plant color or its extract containing one or more of the above.

20. The hair dye according to claim 1, applied to a human body including beards, eyebrows, skins, lips and nails.
